(19) United States
(12) Patent Application Publication
(10) Pub. No.: US 2026/0170812 A1
(43) Pub. Date: Jun. 18, 2026

(54) SYSTEM AND METHODS FOR CHARACTERIZING AND MONITORING INTER-ANATOMICAL REFERENCE RELATIONSHIPS IN MEDICAL IMAGING

(71) Applicant: GE Precision Healthcare LLC, Waukesha, WI (US)

(72) Inventors: Ponnam Mahendhar Goud, Bengaluru (IN); Dattesh Dayanand Shanbhag, Bengaluru (IN); Chitresh Bhushan, Schenectady, NY (US); Tisha Anie Abraham, Bengaluru (IN); Deepa Anand, Bengaluru (IN); Gopal Avinash, San Ramon, CA (US); Sandeep Kaushik, Brookfield, WI (US); Apoorva Anil Agarwal, Bengaluru (IN); Pavan Kumar Kota, Bengaluru (IN)

(21) Appl. No.: 18/983,050

(22) Filed: Dec. 16, 2024

(51) Int. Cl. G06V 10/776; G06V 10/75; G16H 30/20

(52) U.S. Cl. CPC G06V 10/776; G06V 10/751; G16H 30/20; G06V 2201/031

(57) ABSTRACT

A method for monitoring performance of a deployed machine learning model includes receiving a medical image at a clinical site, detecting anatomical landmarks as geometric objects in the medical image using the deployed model, determining spatial relationships between pairs of the detected geometric objects, transmitting the spatial relationships to a remote monitoring system without transmitting the medical image, comparing the spatial relationships against previously determined spatial relationships to identify outliers, generating a performance characterization based on the identified outliers, and responding to deviations from expected model performance by transmitting alerts. The geometric objects include organ masks, segmentation masks, landmark masks, derived planes, and bounding boxes. The spatial relationships include normalized distances between object centers and angles between object orientation vectors. Statistical thresholds enable identification of anomalous relationships while maintaining patient privacy.

### TECHNICAL FIELD

Embodiments of the subject matter disclosed herein relate to medical imaging, and more particularly, to systems and methods for characterizing and monitoring inter-anatomical spatial relationships in medical images processed by machine learning (ML) models.

### BACKGROUND

Machine learning (ML) and deep learning models are increasingly being deployed in medical imaging applications to identify organs, anatomical landmarks, and other anatomical references within three-dimensional (3D) image volumes. These ML models may output various geometry objects, such as organ masks, segmentation masks, landmark masks, planes derived from key landmarks, bounding boxes, or other anatomical reference geometries. The geometry objects may be used for multiple purposes, including guidance during image acquisition, intelligent scan plane placement for consistent imaging, scan reporting to identify pathological conditions, or generating reformatted volumes.

When such ML-powered organ recognition or derived application software is deployed at clinical sites, it is important to track and understand the performance of the ML models to assess reliability and identify situations which might warrant user training or indicate genuine anomalies in the software. However, monitoring ML model performance in deployed clinical settings faces significant challenges. Due to patient privacy concerns and regulatory requirements, patient Protected Health Information (PHI), including imaging data, typically cannot be transmitted back to the software provider from clinical sites without obtaining specific patient consent and Institutional Review Board (IRB) approvals. This makes it difficult to systematically evaluate how well the ML models are performing across different deployment sites and patient populations.

Additionally, while ML models may be validated during development using test datasets, their real-world performance can vary due to differences in patient anatomy, imaging protocols, or other site-specific factors. Any systematic differences between the ML model's predictions and user prescriptions may indicate potential biases that need to be addressed through user training or model updates. However, without access to the underlying clinical imaging data, it is challenging to automatically monitor the reliability of anatomical landmark detection and report any failures, biases, or deviations from expected behavior.

Furthermore, current methods for assessing ML model uncertainty, such as Monte Carlo dropout or test-time augmentation approaches, require multiple inference runs which increases computation time and may not be feasible for real-time clinical use. These methods also may not effectively distinguish between cases where predictions are consistently wrong versus those where predictions are correct but uncertain. Therefore, there remains a need for methods to systematically monitor and characterize ML model performance in clinical deployments without requiring access to protected patient imaging data, while providing meaningful insights about model reliability, potential biases, and genuine anomalies that can guide improvements in both user training and model development.

### SUMMARY

The present disclosure at least partially addresses the issues described above. In one embodiment, a method comprises receiving a medical image at a clinical site, detecting a plurality of anatomical landmarks in the medical image using a deployed machine learning model at the clinical site, determining spatial relationships between pairs of the detected anatomical landmarks, transmitting the determined spatial relationships to a remote monitoring system without transmitting the medical image, comparing the determined spatial relationships against a plurality of previously determined spatial relationships to identify outlier relationships, generating a performance characterization of the deployed machine learning model based on the identified outlier relationships, and responding to the performance characterization indicating a deviation from expected model performance by transmitting an alert to a user device.

In another embodiment, a system comprises a first device located at a clinical site, wherein the first device comprises a first non-transitory memory including a deployed machine learning model and instructions, and a first processor, wherein, when executing the instructions, the first processor causes the first device to receive a medical image, detect a plurality of anatomical landmarks in the medical image using the deployed machine learning model, determine spatial relationships between pairs of the detected anatomical landmarks, and transmit the determined spatial relationships to a second device without transmitting the medical image. The second device is located remotely from the first device, wherein the first device and the second device are communicatively coupled, and wherein the second device comprises a second non-transitory memory including instructions, and a second processor, wherein, when executing the instructions, the second processor causes the second device to receive the determined spatial relationships from the first device, compare the determined spatial relationships against a plurality of previously determined spatial relationships to identify outlier relationships, generate a performance characterization of the deployed machine learning model based on the identified outlier relationships, and respond to the performance characterization indicating a deviation from expected model performance by transmitting an alert to a user device.

In another embodiment, a method for monitoring performance of a deployed machine learning model comprises receiving, at a remote monitoring site, a plurality of spatial interrelationship measurements between anatomical landmarks detected by the deployed machine learning model in a medical image at a deployment site, wherein the spatial interrelationship measurements exclude the medical image and patient identifying information, and wherein the anatomical landmarks comprise one or more geometric objects including organ masks delineating boundaries of anatomical organs, segmentation masks identifying anatomical structures, landmark masks indicating positions of anatomical reference points, planes derived from the anatomical reference points, and bounding boxes encompassing regions of interest, accessing a reference dataset comprising spatial interrelationship measurements between corresponding anatomical landmarks detected in a plurality of training images, for each type of spatial interrelationship measurement between pairs of anatomical landmarks: determining statistical measures from spatial interrelationship measurements from the reference dataset of the type, determining an upper threshold and a lower threshold based on the statistical measures determined from the reference dataset, comparing each received spatial interrelationship measurement against the upper threshold and lower threshold determined for the type of spatial interrelationship measurement, determining a frequency of outlier occurrences for each anatomical landmark by counting a number of times the anatomical landmark appears in spatial interrelationship measurements outside the upper threshold and lower bound threshold, identifying one or more anomalous anatomical landmarks based on the frequency of outlier occurrences exceeding a predetermined frequency threshold, and transmitting an alert identifying the one or more anomalous anatomical landmarks to the deployment site.

In this way, the present disclosure enables monitoring of deployed machine learning model performance without requiring access to protected health information or underlying clinical imaging data. By characterizing and analyzing spatial relationships between detected anatomical landmarks, the system can identify potential model anomalies, biases, or deviations from expected behavior while maintaining patient privacy and regulatory compliance. The spatial relationships serve as statistical surrogates for the underlying clinical data, allowing systematic evaluation of model performance across different deployment sites and patient populations without transmitting sensitive medical images. Additionally, the disclosed approach avoids the computational overhead of uncertainty estimation methods that require multiple model inference runs, making it suitable for real-time clinical applications.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a block diagram of an image processing system, according to an embodiment of the current disclosure;

FIG. 2 is a flowchart illustrating a method for monitoring performance of a trained machine learning model, according to an embodiment of the current disclosure;

FIG. 3 is a flowchart illustrating a method for extracting characteristics of anatomical landmarks, planes, and organs identified in a medical image, according to an embodiment of the current disclosure;

FIG. 4 is a flowchart illustrating a method for determining inter-anatomical spatial relationships between pairs of identified landmarks, planes, and organs, according to an embodiment of the current disclosure;

FIG. 5 is a flowchart illustrating a method for determining a bounding box for normalization of anatomical landmarks, according to an embodiment of the current disclosure;

FIG. 6 is a flowchart illustrating a method for determining anatomical extents based on identified anatomical landmarks, according to an embodiment of the current disclosure;

FIG. 7 is a flowchart illustrating a method for comparing normalized distances and angles against thresholds, according to an embodiment of the current disclosure;

FIG. 8 is a flowchart illustrating another method for comparing normalized distances and angles against thresholds, according to an embodiment of the current disclosure;

FIG. 9 is a flowchart illustrating a method for logging deviations from thresholds, according to an embodiment of the current disclosure;

FIG. 10 is a flowchart illustrating a method for analyzing deviation frequencies of anatomical elements, according to an embodiment of the current disclosure;

FIG. 11 is a flowchart illustrating a method for determining thresholds from a reference dataset, according to an embodiment of the current disclosure;

FIG. 12 is a flowchart illustrating a method for calculating statistical measures and thresholds for inter-anatomical spatial relationships, according to an embodiment of the current disclosure;

FIG. 13 shows an example medical image with anatomical landmarks and planes identified therein, according to an embodiment of the current disclosure;

FIG. 14 shows a plot of signed distances between anatomical landmarks, planes, and organs, according to an embodiment of the current disclosure; and

FIG. 15 shows a plot of angles between anatomical landmarks, planes, and organs, according to an embodiment of the current disclosure.

### DETAILED DESCRIPTION

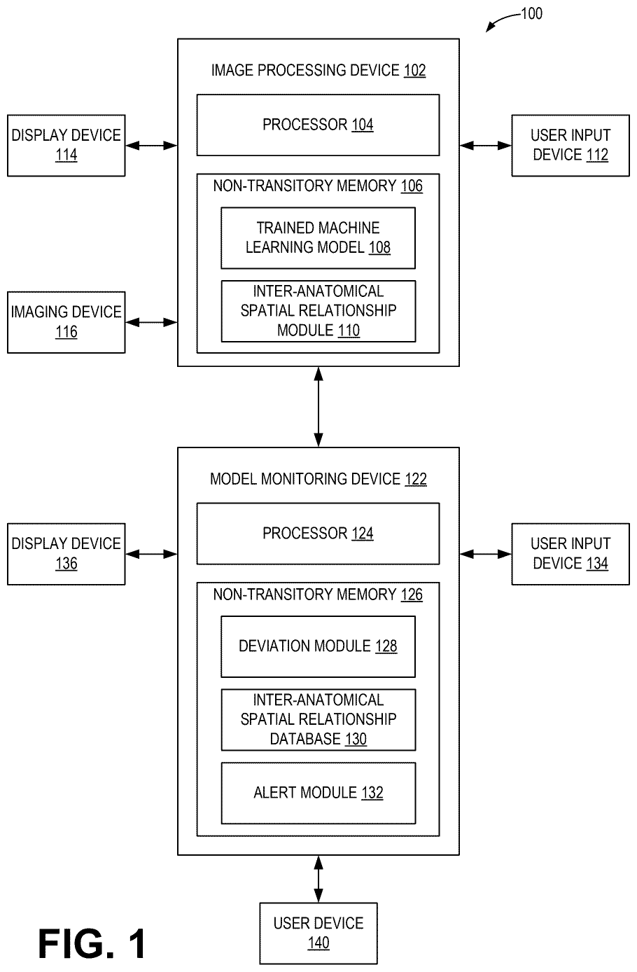

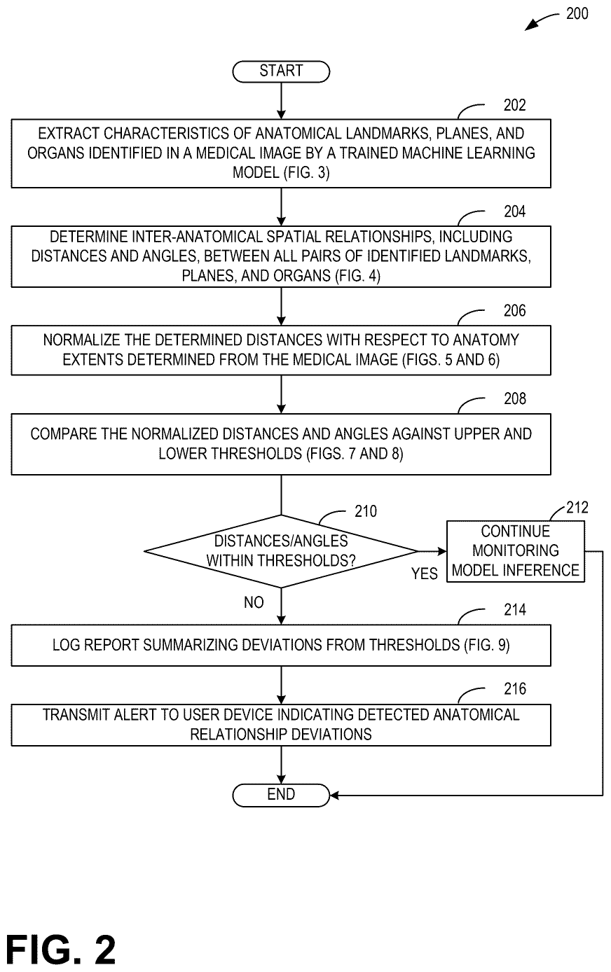

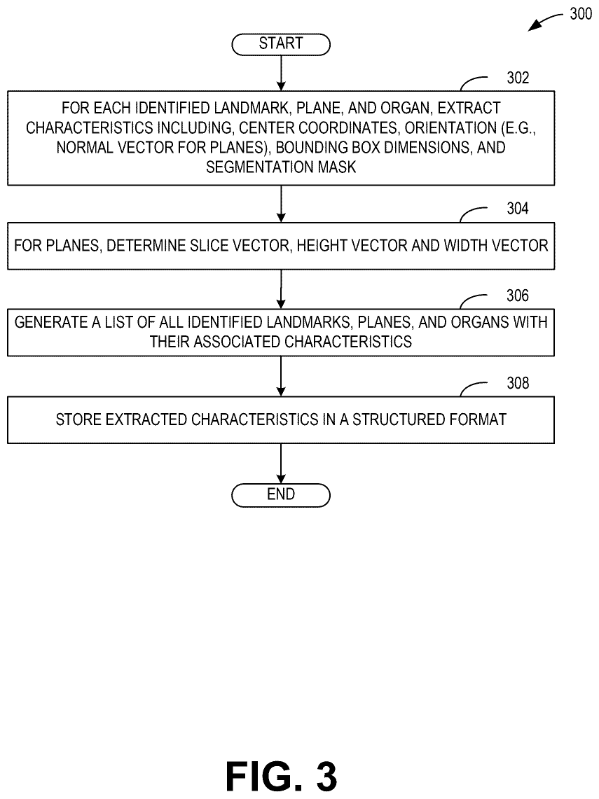

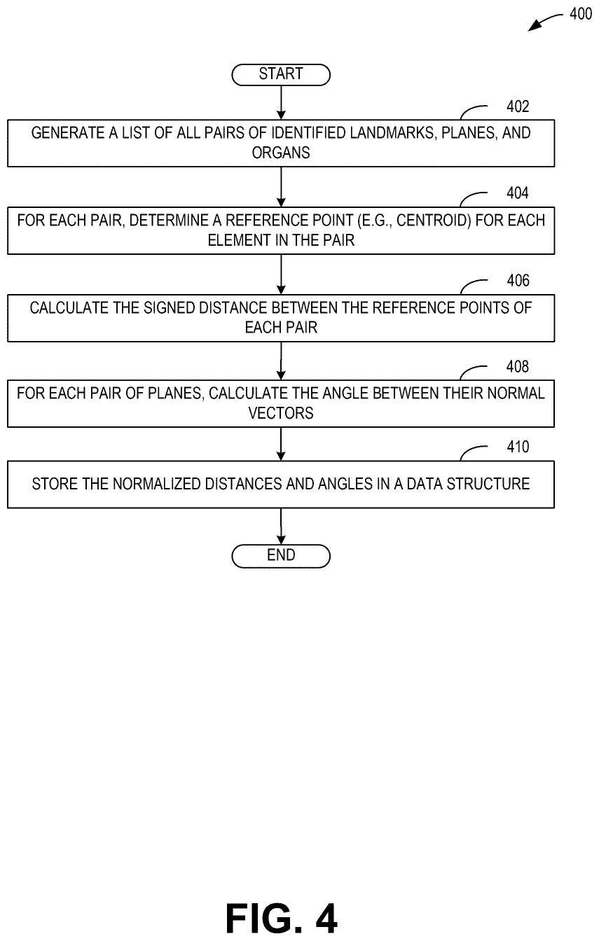

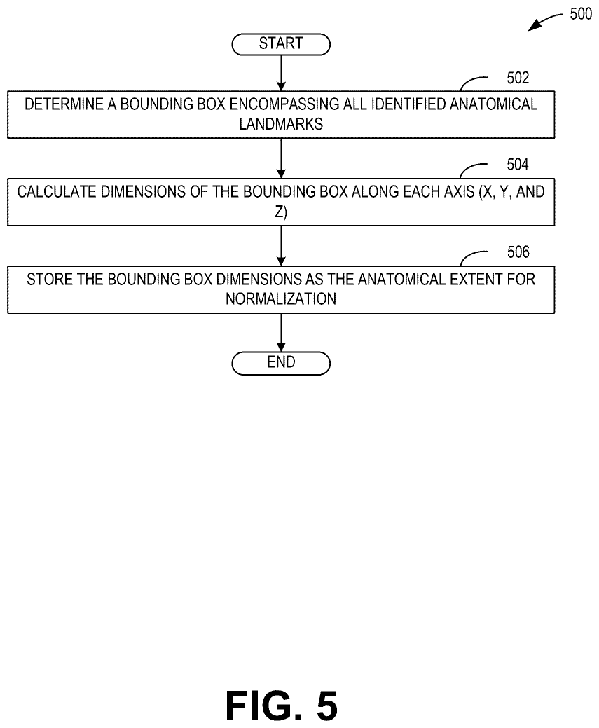

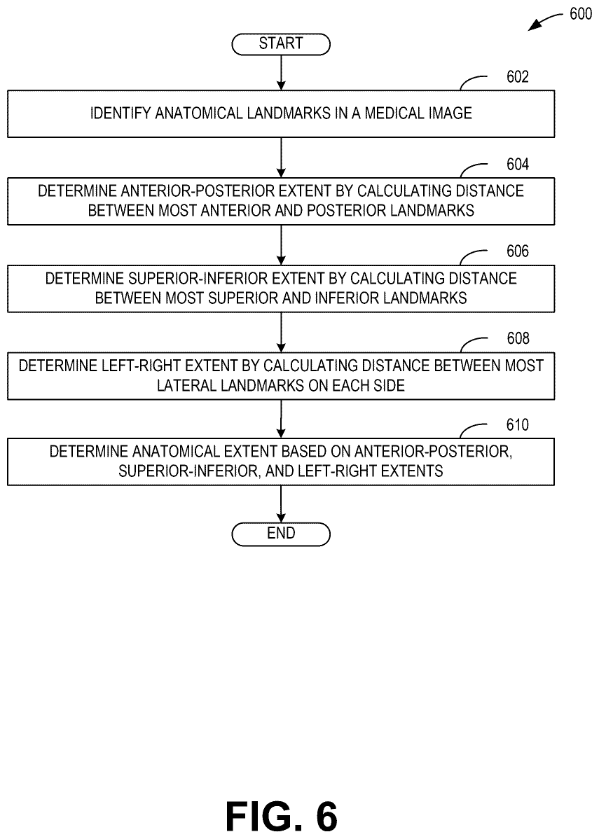

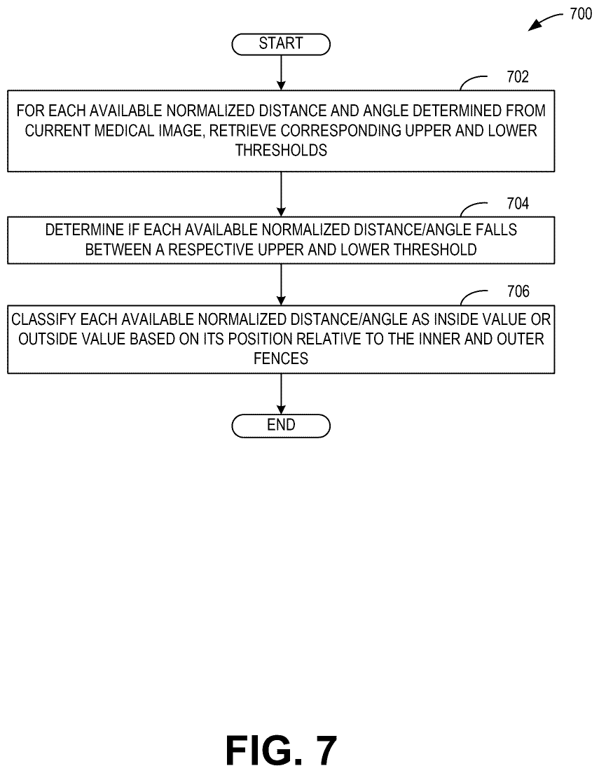

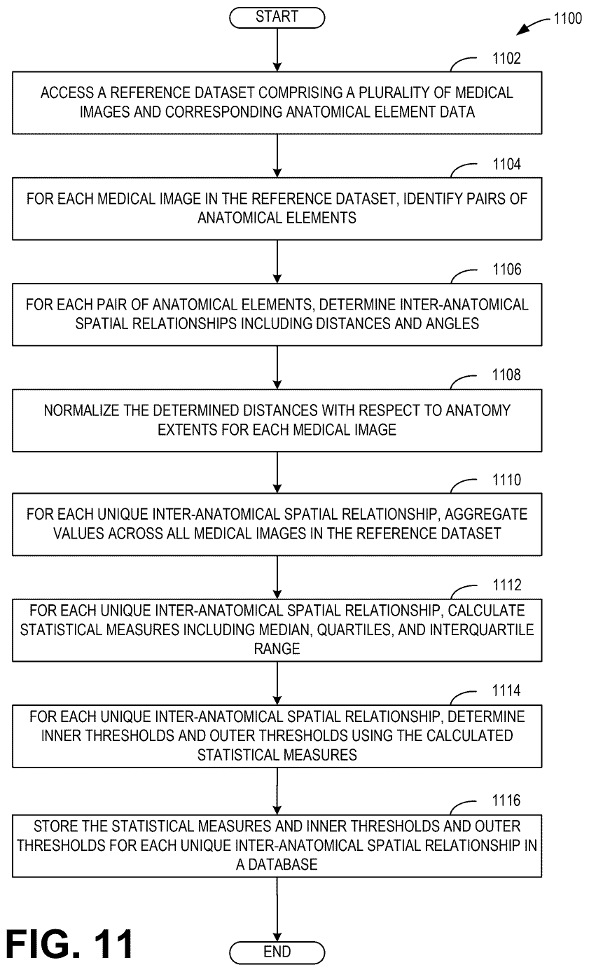

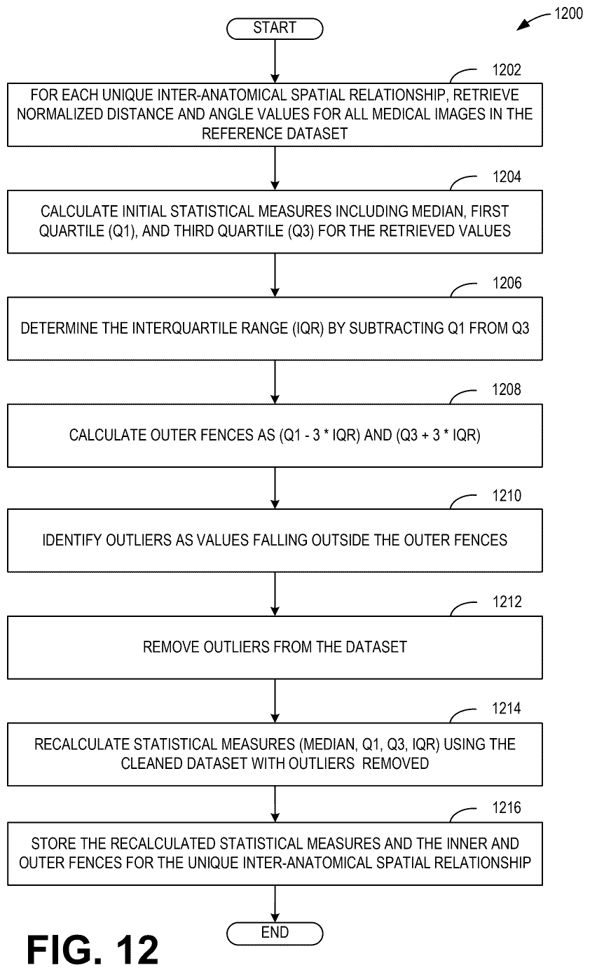

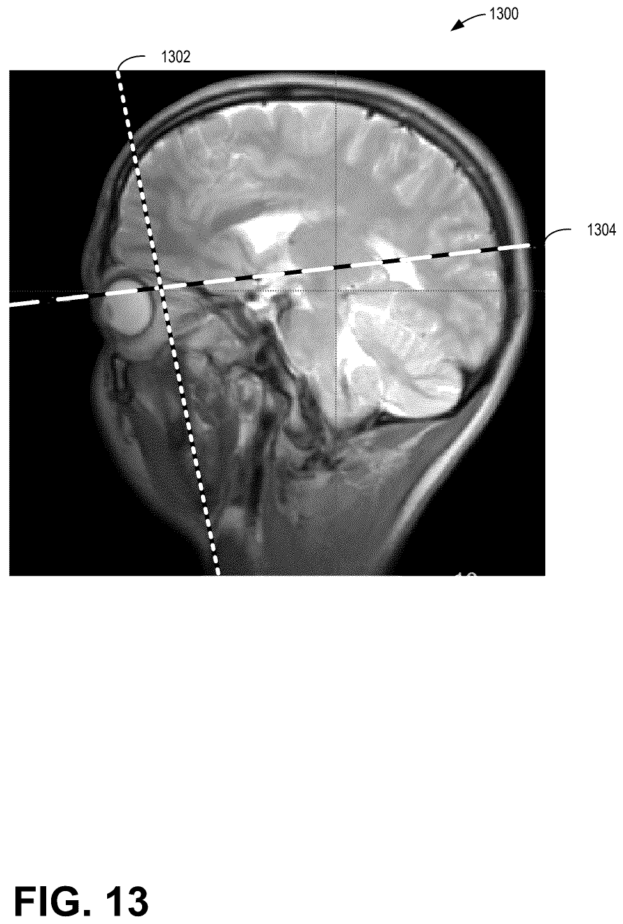

The following description relates to various embodiments of systems and methods for characterizing and monitoring inter-anatomical reference relationships in medical imaging. In particular, the following description provides various approaches for monitoring performance of deployed machine learning models by analyzing spatial relationships between anatomical landmarks, planes, and organs detected in medical images, without requiring access to protected patient imaging data. As shown in FIG. 1, an image processing system may be configured to implement the methods described herein. FIGS. 2-12 show various methods that may be executed by the image processing system of FIG. 1 to monitor performance of deployed machine learning models. Specifically, FIG. 2 shows an overall method for monitoring model performance, while FIGS. 3-4 show methods for extracting and analyzing spatial relationships between detected anatomical elements. FIGS. 5-6 show methods for determining anatomical extents used to normalize measurements across different patient sizes. FIGS. 7-10 show methods for comparing normalized measurements against thresholds and analyzing deviations to characterize model performance. FIGS. 11-12 show methods for establishing thresholds from reference datasets. FIGS. 13-15 show example visualizations of detected anatomical elements and their spatial relationships, including plots demonstrating the statistical distribution of inter-anatomical distances and angles extracted from a reference dataset.

The systems and methods described herein address significant challenges in monitoring the performance of deployed machine learning models in clinical settings. Due to patient privacy concerns and regulatory requirements, patient Protected Health Information (PHI), including imaging data, typically cannot be transmitted back to software providers from clinical sites without obtaining specific patient consent and Institutional Review Board (IRB) approvals. This makes it difficult to systematically evaluate how well machine learning models are performing across different deployment sites and patient populations. Additionally, while ML models may be validated during development using test datasets, their real-world performance can vary due to differences in patient anatomy, imaging protocols, or other site-specific factors.

The present disclosure addresses the above issues by characterizing and analyzing spatial relationships between anatomical elements detected by machine learning models as geometric objects, rather than requiring access to the underlying medical images. These geometric objects may include organ masks delineating boundaries of anatomical organs, segmentation masks identifying anatomical structures, landmark masks indicating positions of anatomical reference points, planes derived from the anatomical reference points, and bounding boxes encompassing regions of interest. The spatial relationships between these geometric objects serve as statistical surrogates for the clinical data, enabling systematic evaluation of model performance while maintaining patient privacy and regulatory compliance. For example, the system analyzes angles between orientation vectors of anatomical planes and normalized distances between centers of organ masks and segmentation masks to identify potential anomalies or biases in model predictions. By normalizing measurements with respect to patient-specific anatomical extents, the system can make meaningful comparisons across different patient sizes and anatomical variations.

Furthermore, the disclosed approach avoids the computational overhead of uncertainty estimation methods that require multiple model inference runs, making it suitable for real-time clinical applications. The system can identify potential model anomalies, biases, or deviations from expected behavior by comparing measured spatial relationships between geometric objects against statistical distributions derived from reference datasets. When deviations are detected, the system can provide targeted feedback about specific anatomical landmarks or relationships that may require additional scrutiny, helping to guide both user training and model improvements. This enables continuous monitoring and improvement of model performance across different deployment sites while ensuring patient privacy and regulatory compliance.

Referring first to FIG. 1, a model monitoring system 100 for monitoring deployed machine learning models using spatial relationship analysis between geometric objects is shown. Model monitoring system 100 may be configured to acquire medical images of an imaging subject using an imaging device 116, process the acquired medical images using an image processing device 102 to detect anatomical landmarks as geometric objects and determine spatial relationships between the detected geometric objects, and monitor performance of deployed machine learning models by analyzing these spatial relationships. The processed information and any detected anomalies may be displayed to a user via display device 114.

Image processing device 102 includes a processor 104 configured to execute machine readable instructions stored in non-transitory memory 106. Non-transitory memory 106 may store the trained machine learning model 108 and the inter-anatomical spatial relationship module 110. The trained machine learning model 108 may include instructions for detecting anatomical landmarks as geometric objects in medical images. In some embodiments, the trained machine learning model 108 may detect organ masks delineating boundaries of anatomical organs, segmentation masks identifying anatomical structures, landmark masks indicating positions of anatomical reference points, planes derived from the anatomical reference points, and bounding boxes encompassing regions of interest. The inter-anatomical spatial relationship module 110 may include instructions for determining angles between orientation vectors of the geometric objects and normalized distances between centers of the geometric objects, wherein the distances are normalized with respect to an anatomical extent determined from the detected anatomical landmarks to account for variations in patient size.

Non-transitory memory 106 may store the trained machine learning model 108 and the inter-anatomical spatial relationship module 110. The trained machine learning model 108 may include instructions for detecting anatomical landmarks in medical images. In some embodiments, the trained machine learning model 108 may detect organ masks, segmentation masks, landmark masks, planes derived from landmarks, or bounding boxes. The inter-anatomical spatial relationship module 110 may include instructions for determining angles between orientation vectors of anatomical landmark planes and normalized distances between centers of the anatomical landmark planes, wherein the distances are normalized with respect to an anatomical extent determined from the detected anatomical landmarks to account for variations in patient size.

Display device 114 may include one or more display devices utilizing virtually any type of technology. The display device 114 may be configured to visually present medical images with detected anatomical landmarks, determined spatial relationships between landmarks, and alerts regarding anomalous spatial relationships. User input device 112 may comprise one or more of a touchscreen, keyboard, mouse, or other device configured to enable a user to interact with the system. Imaging device 116 may include an MRI system, CT scanner, ultrasound system, or other medical imaging device capable of acquiring 2D or 3D medical images.

Model monitoring device 122 includes a processor 124 configured to execute machine readable instructions stored in non-transitory memory 126. Like processor 104, processor 124 may be single or multi-core and may optionally include distributed components configured for coordinated processing. The processor 124 may analyze spatial relationships received from multiple deployment sites to identify systematic biases or anomalies in anatomical landmark detection. In some embodiments, model monitoring device 122 comprises a remote server configured to receive and process data from multiple clinical deployment sites. The remote server includes a database storing spatial relationships between anatomical landmarks detected across multiple deployment sites, along with statistical measures and thresholds derived from these relationships. The remote server further includes a dashboard interface configured to display statistics of model performance characterizations across the multiple clinical sites.

In one embodiment, the model monitoring device 122 receives an indication from the image processing device 102 identifying which anatomical landmarks were selected by a user for prescribing scan planes or other clinical procedures. For example, when a user at a deployment site selects specific anatomical landmarks like the MSP or ACPC plane to prescribe an imaging scan plane, this selection information is transmitted to the model monitoring device 122 along with the spatial relationship measurements via image processing device 102. In some embodiments, if any of the user-selected landmarks match anatomical landmarks that have been identified as anomalous based on their frequency of outlier relationships, the alert module 132 includes a pre-determined warning in the transmitted alert prompting the user to review the prescribed scan plane before proceeding. This enables the system to provide targeted feedback when users attempt to rely on potentially problematic landmark detections for clinical procedures.

Non-transitory memory 126 may store the deviation module 128, inter-anatomical spatial relationship database 130, and alert module 132. The deviation module 128 may include instructions for comparing determined spatial relationships between geometric objects against previously determined spatial relationships to identify outlier relationships. In some embodiments, the deviation module 128 may access pre-determined upper and lower thresholds for each spatial relationship derived from statistical distributions in a reference dataset. The inter-anatomical spatial relationship database 130 may store spatial relationships between geometric objects detected across multiple deployment sites, along with statistical measures and thresholds derived from these relationships.

The alert module 132 may include instructions for generating performance characterizations based on identified outlier relationships between geometric objects and transmitting alerts when deviations from expected model performance are detected. In one embodiment, if a geometric object selected by a user for prescribing a scan plane matches one of the identified anomalous geometric objects (e.g., objects having an outlier frequency exceeding a predetermined frequency threshold), the alert includes a warning prompting the user to review the prescribed scan plane before proceeding with the clinical procedure. The system may also analyze accumulated spatial relationship measurements over time to identify systematic biases in detection of specific geometric objects at the deployment site and provide feedback regarding the identified systematic biases.

Display device 136 may present visualizations of spatial relationship statistics between geometric objects, outlier analyses, and performance trends across different anatomical landmarks and deployment sites. User input device 134 may enable configuration of monitoring parameters and thresholds. User device 140 may comprise a mobile device, computer workstation, or other device configured to receive alerts regarding anomalous spatial relationships or systematic biases detected in anatomical landmark detection.

The model monitoring system 100 enables monitoring of deployed machine learning model performance without requiring access to protected health information or underlying clinical imaging data. By analyzing spatial relationships between detected geometric objects, including organ masks, segmentation masks, landmark masks, derived planes, and bounding boxes, the system can identify potential model anomalies, biases, or deviations from expected behavior while maintaining patient privacy and regulatory compliance. The spatial relationships between these geometric objects serve as statistical surrogates for the underlying clinical data, allowing systematic evaluation of model performance across different deployment sites and patient populations.

Referring to FIG. 2, a flowchart of a method 200 for monitoring deployed ML model performance using inter-anatomical spatial relationships is shown. Method 200 enables monitoring of deployed machine learning model performance without requiring access to protected health information or underlying clinical imaging data by analyzing spatial relationships between detected anatomical landmarks. In one embodiment, a first device at a clinical site receives medical images from an image acquisition device and processes them using a deployed machine learning model stored in a first non-transitory memory, under control of a first processor. The first device determines spatial relationships between detected anatomical landmarks and transmits only these relationships to a second device located at a remote monitoring site, without transmitting the medical images or any patient identifying information. The second device, comprising a second non-transitory memory and second processor, analyzes the received spatial relationships to identify outliers and generate performance characterizations of the deployed model.

At operation 202, the model performance monitoring system extracts characteristics of anatomical landmarks, planes, and organs identified in a medical image by a trained machine learning model. The trained machine learning model detects a plurality of anatomical landmarks in the medical image and outputs various geometry objects, such as organ masks, segmentation masks, landmark masks, planes derived from landmarks, and bounding boxes. For example, in a brain MRI application, the model may detect landmarks including the mid-sagittal plane (MSP), anterior commissure-posterior commissure (ACPC) plane, pituitary gland, optic nerves, temporal lobes, and other anatomical references. In another embodiment, the model identifies organs and anatomical structures in other anatomical regions such as the knee, cardiac, or spine regions.

At operation 204, the model performance monitoring system determines inter-anatomical spatial relationships, also referred to as spatial interrelationship measurements, including distances and angles, between all pairs of identified landmarks, planes, and organs. This includes determining angles between orientation vectors of anatomical landmark planes for each pair of detected anatomical landmarks, and determining distances between centers of the anatomical landmark planes for each pair of detected anatomical landmarks. For example, the angles between planes such as MSP and ACPC, or between the optic nerve planes and temporal lobe planes may be computed. In another embodiment, the spatial relationships are determined between organ masks or bounding boxes by measuring distances and angles between their centers, edges, or other geometric features.

At operation 206, the model performance monitoring system normalizes the determined distances with respect to anatomy extents determined from the medical image. This includes determining a bounding box containing an anatomical region of interest in the medical image, determining dimensions of the bounding box, and dividing each distance measurement by a largest dimension of the bounding box to produce normalized distance measurements that are independent of patient size. In another embodiment, the distances are normalized by determining an extent of an anatomical structure captured in the medical image, computing a reference anatomical size measurement based on the determined extent, and dividing each distance measurement by the reference anatomical size measurement.

At operation 208, the model performance monitoring system compares the normalized distances and angles against upper and lower thresholds. This includes accessing pre-determined upper and lower thresholds for each successfully determined spatial relationship, wherein the thresholds are derived from statistical distributions of previously determined spatial relationships in a reference dataset. The thresholds may be determined by computing statistical quartiles Q1 and Q3 from a distribution of values for each spatial relationship in the reference dataset, wherein Q1 is a 25th percentile and Q3 is a 75th percentile of the distribution of values, and setting the upper threshold to Q3+3(Q3−Q1) and the lower threshold to Q1−3(Q3−Q1). In another embodiment, the thresholds are determined based on median values and interquartile ranges computed from the reference dataset.

In one embodiment, comparing the spatial interrelationship measurements comprises identifying which spatial relationships between pairs of anatomical landmarks were successfully determined based on both landmarks in the pair being detected in the medical image. For example, if a particular landmark is not detected by the deployed machine learning model, spatial interrelationship measurements involving that landmark may not be successfully determined and are excluded from the comparison against previously determined spatial interrelationship measurements. This enables the system to analyze model performance based on available spatial interrelationship measurements while accounting for cases where certain landmarks may not be detected, either due to anatomical variations, image quality issues, or limitations in the model's detection capabilities. The system tracks which landmarks were successfully detected and which spatial relationships could be successfully determined to provide accurate performance characterization focused on spatial interrelationship measurements that can be meaningfully compared against reference data.

At operation 210, the model performance monitoring system determines whether the normalized distances and angles are within their respective thresholds. This decision point evaluates whether any spatial relationships between anatomical landmarks represent potential outliers or deviations from expected relationships by determining if measurements exceed upper thresholds or subceed (i.e., are less than) lower thresholds. If all spatial relationships are within their respective thresholds, neither exceeding upper thresholds nor subceeding lower thresholds, method 200 proceeds to operation 212, where the model performance monitoring system continues monitoring model inference. The system continues to analyze subsequent medical images processed by the deployed machine learning model. Following operation 212 method 200 may end.

However, if at operation 210 any spatial relationships exceed their thresholds, method 200 proceeds to operation 214, where the model performance monitoring system logs a report summarizing the deviations. The report includes a list of anatomical landmark pairs having outlier spatial relationships, and for each outlier spatial relationship, the determined angle and distance value and corresponding upper and lower thresholds. The report may also include a frequency of outlier relationships for each anatomical landmark. In another embodiment, the system determines, for each anatomical landmark, a frequency at which spatial relationships involving that anatomical landmark are identified as outliers, and identifies as anomalous any anatomical landmarks having an outlier frequency exceeding a predetermined frequency threshold.

At operation 216, the model performance monitoring system transmits an alert to a user device indicating detected anatomical relationship deviations. The alert includes an identification of which anatomical landmarks are most frequently involved in outlier relationships, specific angle and distance measurements that exceeded thresholds, and a warning to carefully review any anatomical landmarks identified as anomalous before accepting results from the deployed machine learning model. In another embodiment, if an anatomical landmark selected by a user for prescribing a scan plane matches one of the identified anomalous anatomical landmarks, the alert includes a warning prompting the user to review the scan plane. The system may also analyze accumulated measurements over time to identify systematic biases in detection of specific anatomical landmarks at the deployment site and provide feedback regarding the identified systematic biases.

Following operation 216, method 200 may end. Method 200 provides significant computational advantages by reducing the dimensionality of model performance monitoring from analyzing high-dimensional medical image data to evaluating a compact set of normalized spatial relationships between detected anatomical structures. By transforming complex anatomical detection results into normalized distance and angle measurements, the method enables efficient statistical analysis using lightweight numerical computations rather than computationally intensive image processing operations. Thus for a given processor carrying out the operations described herein, the operations can be carried out faster and/or with less processing requirements than other computer-implements methods not using one or more of the specific actions described herein. For example, the normalization of spatial measurements with respect to anatomy extents makes the analysis robust across patients of different sizes without requiring complex registration or standardization of the underlying medical images. Additionally, the method's use of statistical thresholds derived from reference data distributions provides an automated, quantitative approach for detecting anatomical relationship anomalies that scales efficiently across large numbers of inference results. This computational efficiency enables continuous real-time monitoring of deployed model performance while minimizing processing overhead and storage requirements compared to approaches that analyze raw medical images or require manual review of model outputs.

Referring to FIG. 3, a flowchart of a method 300 for extracting geometric characteristics from identified landmarks in medical imaging data is shown. Method 300 enables compact representation of salient geometric characteristics of anatomical landmarks, planes, and organs identified in medical images by machine learning models, facilitating computationally efficient analysis of spatial relationships.

At operation 302, the image processing system extracts geometric characteristics for each identified landmark, plane, and organ detected in the medical image. In one embodiment, the characteristics include center coordinates of each anatomical element, orientation information such as normal vectors for detected planes, bounding box dimensions encompassing each element, and segmentation masks delineating the boundaries of organs and anatomical structures. For example, in a brain MRI application, the system may extract center coordinates and normal vectors for planes such as the mid-sagittal plane (MSP) and anterior commissure-posterior commissure (ACPC) plane, along with segmentation masks for structures like the pituitary gland and temporal lobes. In another embodiment, the system determines geometric characteristics of organ masks and landmark masks, including centroid locations, principal axes of orientation, and volumetric measurements derived from the segmentation boundaries.

At operation 304, the image processing system determines additional geometric vectors specifically for detected anatomical planes. In one embodiment, this includes calculating a slice vector indicating the primary direction of the plane, a height vector defining the superior-inferior extent, and a width vector defining the lateral extent of each detected plane. For example, when processing brain MRI data, the system may determine these vectors for the MSP to characterize its orientation relative to the scanner coordinate system. In another embodiment, the geometric vectors are computed based on anatomical reference points identified within each plane, such as using the line connecting the anterior and posterior commissures to define the primary direction vector of the ACPC plane.

At operation 306, the image processing system generates a list of all identified landmarks, planes, and organs along with their associated geometric characteristics extracted in operations 302 and 304. In one embodiment, this includes creating data structures that associate each anatomical element with its geometric properties, enabling efficient lookup and comparison of spatial relationships between different elements. For example, the system may generate entries linking each optic nerve landmark with its center coordinates, orientation vectors, and relationships to nearby anatomical structures. In another embodiment, the list includes confidence scores or quality metrics for each detected element, indicating the reliability of the extracted characteristics.

At operation 308, the image processing system stores the extracted characteristics in a structured format suitable for subsequent analysis. In one embodiment, this involves organizing the data into a standardized schema that captures the geometric relationships between anatomical elements while excluding protected health information from the underlying medical images. For example, the system may store normalized distances between landmark pairs and angles between plane normal vectors in a format that enables statistical analysis of anatomical relationships across different patient populations. In another embodiment, the structured format includes hierarchical relationships between anatomical elements, allowing efficient querying of spatial relationships between specific landmarks, planes, or organs of interest.

In this way, method 300 enables computationally efficient extraction and storage of anatomical characteristics from medical images in a format that facilitates monitoring of machine learning model performance through analysis of spatial relationships, while maintaining patient privacy by working with geometric measurements rather than actual image data.

Referring to FIG. 4, a flowchart of a method 400 for determining spatial relationships between anatomical elements is shown. Method 400 may be employed by an image processing system to analyze spatial relationships between anatomical landmarks, planes, and organs detected in medical images by deployed machine learning models, enabling monitoring of model performance without requiring access to protected health information. The determined spatial relationships serve as statistical surrogates for the underlying clinical data, allowing systematic evaluation of model performance across different deployment sites and patient populations while maintaining patient privacy and regulatory compliance.

At operation 402, the image processing system generates a list of all pairs of identified landmarks, planes, and organs. In one embodiment, the system iterates through each detected anatomical element to create pairs with every other detected element, generating a set of relationships to analyze. For example, in a brain MRI application, the system may pair the mid-sagittal plane (MSP) with the anterior commissure-posterior commissure (ACPC) plane, the pituitary gland with each optic nerve, and the temporal lobes with each other. In another embodiment, the system may selectively generate pairs based on predefined anatomical relationships of interest, such as only pairing elements that are expected to maintain consistent spatial relationships across different patients.

At operation 404, the image processing system determines a reference point for each element in the pair. In one embodiment, for planar elements such as the MSP or ACPC plane, the system calculates the centroid or geometric center of the plane within the imaging volume as the reference point. For anatomical organs or structures, the system may determine the center of mass of the segmentation mask or the center of the bounding box containing the structure. In another embodiment, for elongated structures such as nerves or vessels, the system may use endpoints, midpoints, or characteristic points along the structure's path as reference points. For example, when analyzing optic nerve relationships, the system might use the orbital terminus as a reference point.

At operation 406, the image processing system calculates the signed distance between the reference points of each pair. In one embodiment, the system computes the Euclidean distance between the reference points and assigns a sign based on their relative positions along a predetermined anatomical axis, such as anterior-posterior or superior-inferior. For example, when measuring the distance between temporal lobe centers, a positive distance might indicate the second reference point is anterior to the first. In another embodiment, the system may normalize these distances with respect to anatomical extents determined from the medical image to account for variations in patient size. This normalization may involve dividing each distance measurement by a largest dimension of a bounding box containing the anatomical region of interest, or by using a reference anatomical size measurement based on detected anatomical structures.

At operation 408, the image processing system calculates the angle between normal vectors for each pair of planes. In one embodiment, for detected anatomical planes such as MSP and ACPC, the system computes the angle between their orientation vectors using the dot product formula. The system may ensure consistent orientation of the normal vectors based on anatomical conventions, such as having MSP normals point from right to left. In another embodiment, for organs or structures represented by segmentation masks, the system may first fit planes to the structures using principal component analysis or other plane-fitting techniques before calculating inter-plane angles. For example, when analyzing the orientation relationship between optic nerves, the system might fit planes along their primary axes before computing the angle between these fitted planes.

At operation 410, the image processing system stores the normalized distances and angles in a data structure. In one embodiment, the system organizes the measurements in a structured format that associates each spatial relationship with the pair of anatomical elements involved, storing both the raw measurements and their normalized values. The data structure may also include metadata such as confidence scores for the detected elements and flags indicating whether specific relationships could not be determined due to missing detections. In another embodiment, the system may store the measurements in a format optimized for statistical analysis, such as arrays or matrices that facilitate efficient computation of statistical measures and comparison against reference distributions. This structured storage enables subsequent analysis of model performance while maintaining patient privacy, as only the geometric relationships between anatomical elements are retained without the underlying medical images.

Following operation 410, method 400 may end. In this way, method 400 enables efficient computation and storage of spatial relationships between anatomical elements while minimizing memory requirements by storing only the salient geometric measurements rather than entire medical images or the entire detected landmark. The normalized measurements and structured storage format reduce computational overhead by eliminating the need for repeated normalization calculations during statistical analysis. Additionally, by computing distances and angles between reference points rather than comparing entire segmentation masks or volumes, the method achieves significant computational efficiency compared to traditional anatomical comparison approaches. The method's focus on geometric relationships also enables rapid identification of potential model anomalies without requiring computationally expensive uncertainty estimation methods that demand multiple model inference runs.

Referring to FIG. 5, a flowchart of a method 500 for determining anatomical extent for normalization is shown. Method 500 enables normalization of spatial measurements between anatomical landmarks with respect to patient-specific anatomy size, allowing meaningful comparison of inter-anatomical relationships across different patient populations while maintaining patient privacy and regulatory compliance.

At operation 502, the image processing system determines a bounding box encompassing all identified anatomical landmarks detected in the medical image. In one embodiment, the system employs a trained machine learning model to analyze the medical image and output a bounding box that encompasses the anatomical region of interest. The machine learning model may be trained on a large dataset of medical images with annotated bounding boxes to learn the appropriate extent and positioning of the bounding box based on the detected anatomical landmarks and structures. For example, when processing brain MRI data, the machine learning model may output a bounding box that appropriately encompasses key neurological structures including the mid-sagittal plane (MSP), anterior commissure-posterior commissure (ACPC) plane, pituitary gland, optic nerves, temporal lobes, and other detected anatomical references. In another embodiment, the system may employ multiple specialized machine learning models trained for different anatomical regions, enabling region-specific bounding box determination optimized for particular anatomical structures or imaging protocols.

At operation 504, the image processing system calculates dimensions of the bounding box along each axis (x, y, and z). In one embodiment, this includes computing the absolute distances between the minimum and maximum extents of the machine learning model-generated bounding box along each orthogonal axis of the imaging coordinate system to determine the anterior-posterior, superior-inferior, and left-right dimensions. For example, the system may calculate the anterior-posterior extent as the distance between the anterior and posterior faces of the bounding box, the superior-inferior extent as the distance between the superior and inferior faces, and the left-right extent as the distance between the lateral faces. In another embodiment, the system may calculate additional geometric properties of the bounding box, such as its volume, surface area, or diagonal lengths, to provide multiple options for normalizing spatial measurements based on different anatomical size metrics.

At operation 506, the image processing system stores the bounding box dimensions as the anatomical extent for normalization. In one embodiment, the system saves the dimensions in a structured format that associates them with the specific medical image or patient case, while excluding protected health information, enabling their use for normalizing spatial relationships between anatomical landmarks. For example, when analyzing distances between detected landmarks, each distance measurement may be divided by the largest dimension of the stored bounding box to produce normalized measurements that are independent of patient size. In another embodiment, the system may store multiple normalization references derived from the bounding box dimensions, such as the geometric mean of the dimensions or the cube root of the bounding box volume, allowing selection of the most appropriate normalization metric for different types of spatial measurements.

Following operation 506, method 500 may end. The stored anatomical extent enables subsequent normalization of spatial relationships between detected anatomical landmarks, facilitating statistical analysis of anatomical relationships across different patient populations.

Referring to FIG. 6, a flowchart of a method 600 for determining anatomical extent from medical image data is shown. Method 600 enables normalization of spatial measurements between anatomical landmarks with respect to patient-specific anatomy size, allowing meaningful comparison of inter-anatomical relationships across different patient populations while maintaining patient privacy and regulatory compliance.

At operation 602, the image processing system identifies anatomical landmarks in a medical image using a trained machine learning model. In one embodiment, the trained machine learning model detects a plurality of anatomical landmarks in the medical image and outputs various geometry objects, such as organ masks, segmentation masks, landmark masks, planes derived from key landmarks, and bounding boxes. For example, in a brain MRI application, the model may detect landmarks including the MSP, ACPC plane, pituitary gland, optic nerves, temporal lobes, and other anatomical references. In another embodiment, the model identifies organs and anatomical structures in other anatomical regions such as the knee, cardiac, or spine regions.

At operation 604, the image processing system determines the anterior-posterior extent by calculating the distance between the most anterior and posterior landmarks identified in the medical image. In one embodiment, this includes analyzing the positions of all detected landmarks in the anterior-posterior direction and identifying the landmarks with the maximum and minimum anterior-posterior coordinates to determine the overall anterior-posterior anatomical extent. For example, when processing brain MRI data, the system may measure the distance between the most anterior point of the frontal lobe and the most posterior point of the occipital lobe. In another embodiment, the anterior-posterior extent is determined by measuring the distance between faces of a bounding box containing all detected anatomical landmarks.

At operation 606, the image processing system determines the superior-inferior extent by calculating the distance between the most superior and inferior landmarks identified in the medical image. In one embodiment, this includes analyzing the positions of all detected landmarks in the superior-inferior direction and identifying the landmarks with the maximum and minimum superior-inferior coordinates to determine the overall superior-inferior anatomical extent. For example, when processing brain MRI data, the system may measure the distance between the most superior point of the cortex and the most inferior point of the brainstem. In another embodiment, the superior-inferior extent is determined by measuring the distance between the superior and inferior faces of a bounding box containing all detected anatomical landmarks.

At operation 608, the image processing system determines the left-right extent by calculating the distance between the most lateral landmarks on each side of the medical image. In one embodiment, this includes analyzing the positions of all detected landmarks in the left-right direction and identifying the landmarks with the maximum left and right coordinates to determine the overall left-right anatomical extent. For example, when processing brain MRI data, the system may measure the distance between the most lateral points of the left and right temporal lobes. In another embodiment, the left-right extent is determined by measuring the distance between the lateral faces of a bounding box containing all detected anatomical landmarks.

At operation 610, the image processing system determines the anatomical extent based on the anterior-posterior, superior-inferior, and left-right extents calculated in operations 604-608. In one embodiment, the system stores the dimensions in a structured format that associates them with the specific medical image, enabling their use for normalizing spatial relationships between anatomical landmarks. For example, when analyzing distances between detected landmarks, each distance measurement may be divided by the largest dimension of the stored anatomical extent to produce normalized measurements that are independent of patient size. In another embodiment, the system may store multiple normalization references derived from the anatomical extent dimensions, such as the geometric mean of the dimensions or the cube root of the bounding box volume, allowing selection of the most appropriate normalization metric for different types of spatial measurements.

Following operation 610, method 600 may end. The stored anatomical extent enables subsequent normalization of spatial relationships between detected anatomical landmarks, facilitating statistical analysis of anatomical relationships across different patient populations.

Referring to FIG. 7, a flowchart of a method 700 for comparing normalized distances and angles against thresholds in medical image analysis is shown. Method 700 enables efficient evaluation of spatial relationships between anatomical landmarks detected by deployed machine learning models by comparing each normalized measurement against pre-computed upper and lower thresholds, rather than requiring computationally expensive comparisons against an entire reference dataset. The thresholds are derived from statistical distributions of previously determined spatial relationships, allowing rapid identification of potential outliers while maintaining patient privacy and regulatory compliance by analyzing normalized geometric measurements rather than actual medical image data. This computationally efficient approach reduces the dimensionality of model performance monitoring from analyzing high-dimensional medical image data to evaluating a compact set of threshold comparisons.

At operation 702, the image processing system retrieves corresponding upper and lower thresholds for each available normalized distance and angle determined from the current medical image. In one embodiment, the system accesses pre-determined upper and lower thresholds derived from statistical distributions of previously determined spatial relationships in a reference dataset. The thresholds may be determined by computing statistical quartiles Q1 and Q3 from a distribution of values for each spatial relationship in the reference dataset, wherein Q1 is a 25th percentile and Q3 is a 75th percentile of the distribution of values, and setting the upper threshold to Q3+3(Q3−Q1) and the lower threshold to Q1−3(Q3−Q1). In another embodiment, the thresholds are determined based on median values and interquartile ranges computed from the reference dataset, with the upper and lower thresholds representing statistical bounds for expected variations in anatomical relationships.

At operation 704, the image processing system determines if each available normalized distance and angle falls between its respective upper and lower threshold. In one embodiment, the system compares each normalized measurement against the retrieved thresholds to identify potential outliers or deviations from expected anatomical relationships. For example, when analyzing angles between orientation vectors of anatomical landmark planes, the system evaluates whether each angle measurement falls within the statistically determined acceptable range for that specific pair of landmarks. In another embodiment, the system analyzes normalized distances between centers of anatomical landmarks, comparing each distance measurement against threshold values that account for expected variations in patient anatomy while remaining independent of patient size due to the normalization.

At operation 706, the image processing system classifies each available normalized distance and angle as an inside value or outside value based on its position relative to the upper and lower thresholds. In one embodiment, measurements falling between the thresholds are classified as inside values, representing expected anatomical relationships, while measurements falling outside the thresholds are classified as outside values, indicating potential anomalies in the machine learning model's landmark detection. For example, if the angle between the MSP and ACPC plane exceeds the upper threshold, it would be classified as an outside value warranting further investigation.

Following operation 706, method 700 may end. In this way, method 700 provides robust statistical analysis of anatomical relationships by using only pre-computed upper and lower thresholds for each unique inter-anatomical relationship, enabling efficient comparison of spatial measurements without requiring access to or storage of the entire reference dataset. The method's use of statistically derived thresholds for each specific anatomical relationship pair allows rapid evaluation of model performance through simple numerical comparisons, rather than requiring computationally expensive comparisons against a full distribution of reference measurements. By transforming complex anatomical detection results into a compact set of thresholds customized for each unique spatial relationship, the method enables lightweight monitoring of deployed model performance while minimizing processing overhead and storage requirements compared to approaches that maintain complete reference distributions.

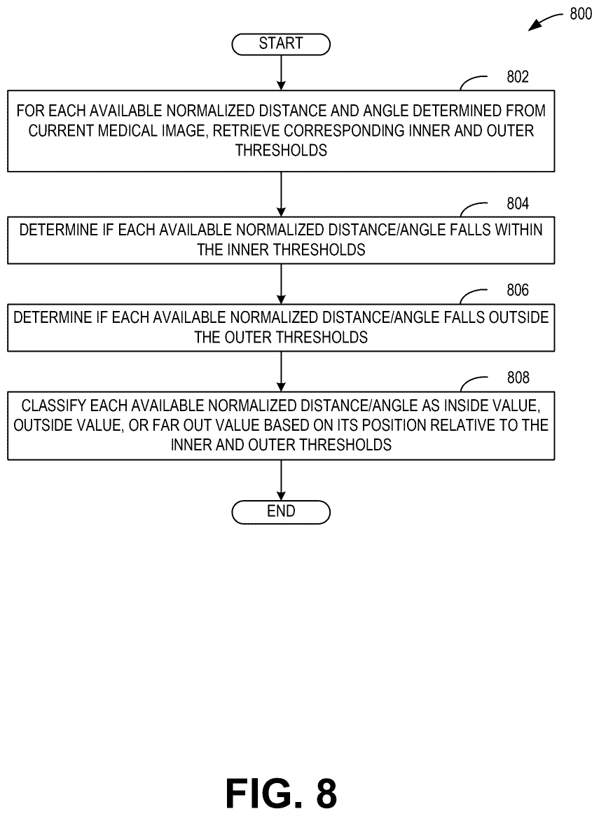

Referring to FIG. 8, a flowchart of a method 800 for classifying inter-anatomical spatial relationships in medical images is shown. Method 800 enables systematic evaluation of spatial relationships between anatomical landmarks detected by machine learning models by comparing normalized distances and angles against statistically-derived thresholds. The method classifies each spatial relationship measurement as an inside value, outside value, or far out value based on its position relative to inner and outer thresholds determined from reference datasets, providing a quantitative approach for identifying potential anomalies in anatomical landmark detection.

At operation 802, the image processing system retrieves corresponding inner and outer thresholds for each available normalized distance and angle determined from the current medical image. In one embodiment, the system accesses pre-determined inner thresholds, comprising an inner upper threshold and an inner lower threshold, where the inner upper threshold is set to Q3+1.5(Q3−Q1) and the inner lower threshold is set to Q1−1.5(Q3−Q1), where Q1 is the 25th percentile and Q3 is the 75th percentile of the distribution of values from a reference dataset. The system also accesses pre-determined outer thresholds, comprising an outer upper threshold and an outer lower threshold, where the outer upper threshold is set to Q3+3(Q3−Q1) and the outer lower threshold is set to Q1−3(Q3−Q1). In another embodiment, the inner and outer thresholds are determined based on median values and interquartile ranges computed from the reference dataset.

At operation 804, the image processing system determines if each available normalized distance and angle falls within the inner thresholds. In one embodiment, the system compares each spatial relationship measurement against its corresponding inner upper and inner lower thresholds to identify measurements that represent typical anatomical relationships. For example, when analyzing the angle between the MSP and ACPC plane, the system may check if the measured angle falls between the inner fences defined by Q1−1.5(Q3−Q1) and Q3+1.5(Q3−Q1). In another embodiment, the system may apply different inner threshold criteria based on the specific type of spatial relationship being evaluated.

At operation 806, the image processing system determines if each available normalized distance and angle falls outside the outer thresholds. In one embodiment, the system identifies extreme outliers by comparing each spatial relationship measurement against its corresponding outer upper and outer lower thresholds, which represent the boundaries of anatomically plausible relationships. For example, when evaluating the normalized distance between optic nerve landmarks, measurements beyond either the outer upper threshold of Q3+3(Q3−Q1) or the outer lower threshold of Q1−3(Q3−Q1) may indicate potential errors in landmark detection. In another embodiment, the system may consider the frequency of outer threshold violations for specific landmarks to identify systematic biases in the machine learning model's detection capabilities.

At operation 808, the image processing system classifies each available normalized distance and angle as an inside value, outside value, or far out value based on its position relative to the inner and outer thresholds. In one embodiment, measurements falling within the inner thresholds (between the inner upper and inner lower thresholds) are classified as inside values representing typical anatomical relationships, measurements between the inner and outer thresholds are classified as outside values indicating potential anomalies, and measurements beyond the outer thresholds are classified as far out values suggesting significant deviations requiring immediate attention. For example, if the angle between temporal lobe planes exceeds the outer upper threshold or falls below the outer lower threshold, it may be classified as a far out value triggering an alert to review the landmark detection results. In another embodiment, the classification may include confidence scores based on how far measurements deviate from the inner and outer thresholds.

Following operation 808, method 800 may end. Method 800 provides significant technical advantages by implementing a hierarchical classification approach using inner and outer thresholds that enables more nuanced monitoring of anatomical relationship deviations. By classifying spatial relationships into three categories-inside values within inner thresholds representing typical anatomical relationships, outside values between inner and outer thresholds indicating potential anomalies requiring review, and far out values beyond outer thresholds suggesting significant deviations-the method enables prioritization of cases requiring manual review while reducing false positives. The dual threshold approach provides greater sensitivity in detecting subtle deviations from expected anatomical relationships compared to single threshold approaches, while maintaining specificity by requiring measurements to exceed outer thresholds before triggering alerts. This hierarchical classification scheme also enables efficient allocation of review resources by allowing different response protocols for outside values versus far out values, improving the overall efficiency of model performance monitoring across deployment sites.

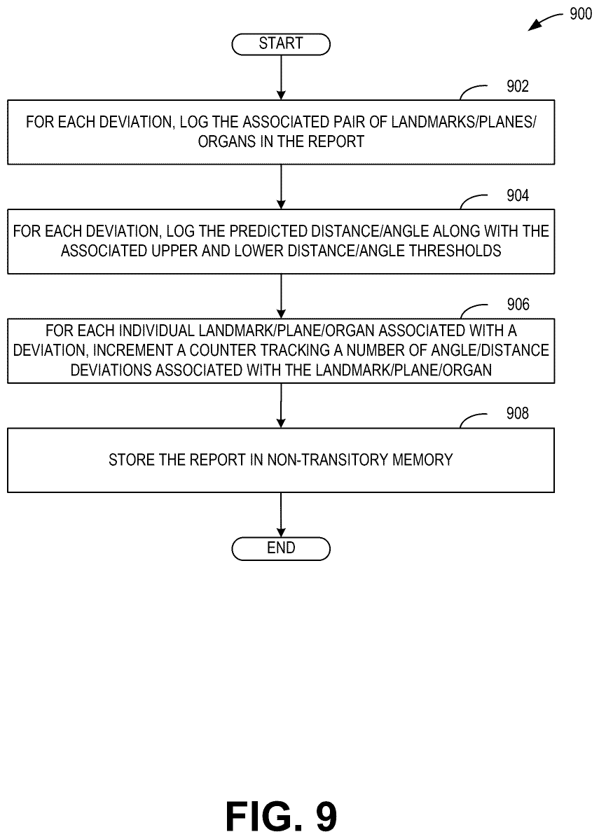

Referring to FIG. 9, a flowchart of a method 900 for logging deviations when spatial relationships between anatomical landmarks fall outside expected thresholds is shown. Method 900 enables tracking and reporting of anomalous spatial relationships detected between anatomical landmarks, planes, and organs identified in medical images by deployed machine learning models. The logged deviation information provides detailed records of which anatomical elements are most frequently involved in outlier relationships, allowing targeted investigation of potential model performance issues while maintaining patient privacy by working only with geometric measurements rather than actual medical image data.

At operation 902, the image processing system logs the associated pair of landmarks, planes, or organs in the report for each deviation. In one embodiment, this includes creating entries in the report that identify which specific anatomical elements were involved in spatial relationships exceeding the predetermined thresholds. For example, if the angle between the MSP and ACPC plane exceeds expected bounds, the system would log both the MSP and ACPC plane as the anatomical pair involved in the deviation. In another embodiment, the system may organize the logged pairs hierarchically based on anatomical relationships, grouping deviations involving related structures like the optic nerves or temporal lobes together to facilitate pattern analysis.

At operation 904, the image processing system logs the predicted distance or angle measurement along with the associated upper and lower thresholds for each deviation. In one embodiment, this includes recording the specific normalized distance or angle value that exceeded bounds, along with the corresponding statistical thresholds derived from the reference dataset against which it was compared. For example, the system may log that the normalized distance between optic nerve landmarks was 0.8, exceeding the upper threshold of 0.6 determined from the reference data distribution. In another embodiment, the system may also record the magnitude of the deviation, calculated as the difference between the measured value and the nearest threshold, to help quantify the magnitude of each anomalous relationship.

At operation 906, the image processing system increments a counter tracking the number of angle and distance deviations associated with each individual landmark, plane, or organ involved in a deviation. In one embodiment, this includes maintaining separate counters for each anatomical element that track how frequently it appears in outlier relationships by counting a number of times that element appears in spatial relationships exceeding the predetermined thresholds. For example, if the right optic nerve is involved in multiple outlier relationships with different landmarks, its counter would be incremented each time to maintain a count of its outlier occurrences. In another embodiment, the system may maintain separate counters for angle deviations versus distance deviations, enabling more granular analysis of whether specific landmarks tend to have orientation issues versus positioning issues by counting the number of times each type of deviation occurs.

At operation 908, the image processing system stores the report in non-transitory memory. In one embodiment, this includes saving the compiled deviation information in a structured format that associates each anatomical element with its deviation frequency and specific outlier relationships, while excluding protected health information from the underlying medical images. For example, the system may store the report as a JSON structure containing nested objects for each anatomical landmark and its associated deviation data. In another embodiment, the system may organize the stored reports by deployment site and time period to enable trend analysis of model performance across different clinical settings and patient populations.

Following operation 908, method 900 may end. Method 900 provides technical advantages by transforming complex anatomical detection results into a compact, analyzable format focused on deviation patterns. By tracking and organizing outlier relationships by anatomical element, the method enables efficient identification of systematic issues in landmark detection while minimizing storage requirements compared to approaches that store raw medical images. The method's structured logging approach also facilitates automated analysis of deviation patterns across multiple deployment sites while maintaining patient privacy through the use of normalized geometric measurements.

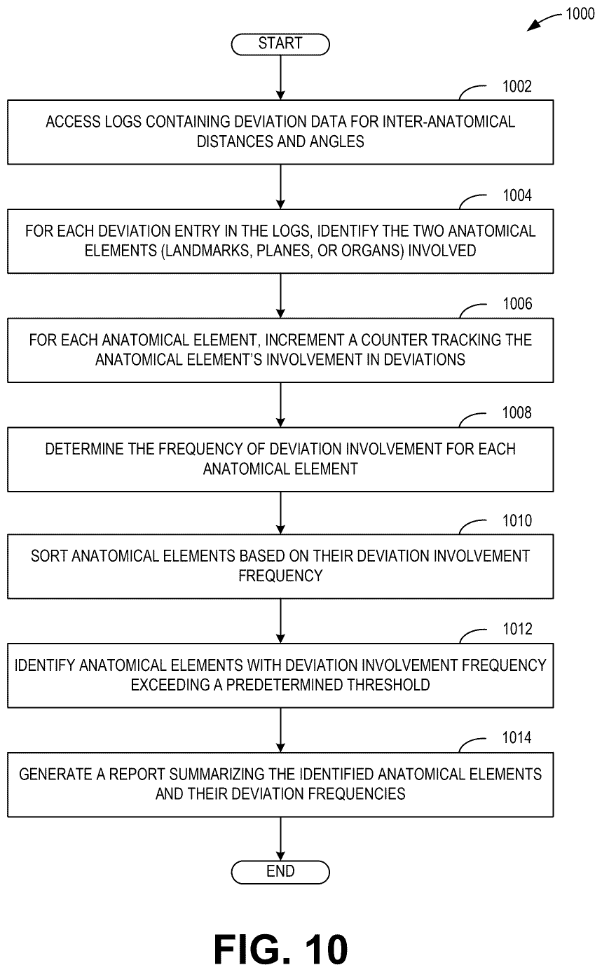

Referring to FIG. 10, a flowchart of a method 1000 for analyzing deviation patterns in anatomical landmark detection to identify potentially problematic landmarks is shown. Method 1000 enables systematic analysis of outlier relationships between anatomical landmarks detected by deployed machine learning models to identify landmarks that are frequently involved in anomalous spatial relationships, indicating potential inaccuracies in the model's detection capabilities for those specific anatomical elements.

At operation 1002, the image processing system accesses logs containing deviation data for inter-anatomical distances and angles. In one embodiment, these logs include reports generated when normalized spatial relationships between anatomical landmarks exceed predetermined upper and lower thresholds derived from reference datasets. The logs may contain entries listing pairs of anatomical landmarks having outlier relationships, along with their specific angle and distance measurements and corresponding threshold values. In another embodiment, the logs may include structured data associating each anatomical element with geometric characteristics and spatial relationships that were identified as anomalous, while excluding protected health information from the underlying medical images.

At operation 1004, the image processing system identifies the two anatomical elements involved for each deviation entry in the logs. In one embodiment, this involves parsing each log entry to extract the pair of anatomical landmarks, planes, or organs associated with each outlier spatial relationship. For example, if an angle between the MSP and ACPC plane exceeds expected bounds, both the MSP and ACPC plane would be identified as the anatomical elements involved in that deviation. In another embodiment, the system may organize the identified pairs hierarchically based on anatomical relationships, grouping deviations involving related structures like optic nerves or temporal lobes together to facilitate pattern analysis.

At operation 1006, the image processing system increments a counter tracking the anatomical element's involvement in deviations for each anatomical element. In one embodiment, this includes maintaining separate counters for each unique anatomical landmark, plane, or organ that counts a number of times it appears in outlier relationships with other anatomical elements. For example, if the right optic nerve is involved in multiple outlier relationships with different landmarks, its counter would be incremented to track the exact number of times that landmark appears in relationships exceeding the thresholds. In another embodiment, the system may maintain separate counters for angle deviations versus distance deviations, enabling more granular analysis of whether specific landmarks tend to have orientation issues versus positioning issues by maintaining precise counts of each type of deviation occurrence.

At operation 1008, the image processing system determines the frequency of deviation involvement for each anatomical element. In one embodiment, this involves calculating, for each anatomical landmark, a ratio of the number of times that landmark appears in outlier relationships to the total number of spatial relationships involving that landmark. This normalized frequency accounts for landmarks that may be involved in different numbers of total relationships. In another embodiment, the system may calculate separate frequencies for different types of deviations, such as the frequency of angle outliers versus distance outliers, or the frequency of exceeding upper thresholds versus lower thresholds.

At operation 1010, the image processing system sorts anatomical elements based on their deviation involvement frequency. In one embodiment, this includes organizing the anatomical landmarks in descending order of their outlier frequencies to identify which landmarks are most frequently involved in anomalous spatial relationships. For example, if the pituitary gland is involved in outlier relationships 30% of the time while other landmarks average 5%, it would be sorted to the top of the list. In another embodiment, the system may perform separate sorting for different types of deviations or different anatomical regions, enabling focused analysis of specific aspects of model performance.

At operation 1012, the image processing system identifies anatomical elements with deviation involvement frequency exceeding a predetermined threshold. In one embodiment, this involves comparing each landmark's outlier frequency against a threshold frequency value to identify landmarks that are anomalously often involved in outlier relationships. The threshold may be determined based on statistical analysis of typical deviation frequencies across all landmarks. In another embodiment, the system may employ different thresholds for different types of anatomical elements or different types of spatial relationships, accounting for varying degrees of expected variability in different anatomical relationships.

At operation 1014, the image processing system generates a report summarizing the identified anatomical elements and their deviation frequencies. In one embodiment, the report includes a list of anatomical landmarks identified as anomalous, along with their specific outlier frequencies and the types of spatial relationships in which they most commonly deviate. For example, the report might indicate that the temporal lobe landmarks are involved in outlier relationships 25% of the time, primarily in angular relationships with other landmarks. In another embodiment, the report may include visualizations of deviation patterns, statistical analyses of outlier frequencies, and specific recommendations for reviewing and validating the machine learning model's detection capabilities for the identified problematic landmarks.

Following operation 1014, method 1000 may end. In this way, method 1000 enables systematic identification of potentially problematic anatomical landmarks by analyzing patterns in spatial relationship deviations across multiple cases. By tracking and analyzing the frequency with which different anatomical elements are involved in outlier relationships, the method helps identify specific landmarks or anatomical structures that may require additional attention or refinement in the machine learning model's detection capabilities.

Referring to FIG. 11, a flowchart of a method 1100 for generating statistical measures of inter-anatomical spatial relationships from a reference dataset of medical images is shown. Method 1100 enables generation of statistical thresholds for monitoring deployed machine learning model performance by analyzing spatial relationships between anatomical landmarks, planes, and organs detected in medical images, while maintaining patient privacy and regulatory compliance.

At operation 1102, the image processing system accesses a reference dataset comprising a plurality of medical images and corresponding anatomical element data. In one embodiment, the reference dataset includes medical images from multiple clinical sites along with detected anatomical landmarks, planes, and organs identified in each image by a trained machine learning model. For example, in cardiac MRI applications, the anatomical elements may include the four-chamber plane, short-axis plane, aortic valve plane, left and right ventricles, coronary arteries, and other cardiovascular structures. In another embodiment, the reference dataset may include manually validated anatomical element detections to ensure the statistical measures are derived from accurate landmark identifications.

At operation 1104, the image processing system identifies pairs of anatomical elements for each medical image in the reference dataset. In one embodiment, the system iterates through each detected anatomical element to create pairs with every other detected element, generating a set of relationships to analyze. For example, the system may pair the four-chamber plane with the short-axis plane, the aortic valve with each ventricle, and the left and right ventricles with each other. In another embodiment, the system may selectively generate pairs based on predefined anatomical relationships of interest, such as only pairing elements that are expected to maintain consistent spatial relationships across different patients.

At operation 1106, the image processing system determines inter-anatomical spatial relationships including distances and angles for each pair of anatomical elements. In one embodiment, for planar elements such as the four-chamber plane or short-axis plane, the system calculates angles between their orientation vectors using the dot product formula and determines distances between their centroids. For anatomical organs or structures represented by segmentation masks, the system may first fit planes to the structures using principal component analysis before computing inter-plane angles. In another embodiment, the system calculates signed distances between reference points of each pair, assigning a sign based on their relative positions along predetermined anatomical axes such as anterior-posterior or superior-inferior.

At operation 1108, the image processing system normalizes the determined distances with respect to anatomy extents for each medical image. In one embodiment, this includes determining a bounding box containing the anatomical region of interest, calculating dimensions along each axis (x, y, and z), and dividing each distance measurement by the largest dimension to produce normalized measurements that are independent of patient size. In another embodiment, the system determines anatomical extents by calculating distances between the most anterior-posterior, superior-inferior, and lateral landmarks identified in the medical image, then uses these anatomical extents to normalize the spatial measurements.

At operation 1110, the image processing system aggregates values across all medical images in the reference dataset for each unique inter-anatomical spatial relationship. In one embodiment, the system collects all normalized distance measurements and angles for each specific pair of anatomical elements, creating distributions of values that characterize the expected spatial relationships. For example, the system may aggregate all measurements of the angle between the four-chamber plane and short-axis plane across the reference dataset to establish the normal range of this relationship. In another embodiment, the system may organize the aggregated measurements hierarchically based on anatomical regions or types of relationships to facilitate efficient statistical analysis.

At operation 1112, the image processing system calculates statistical measures including median, quartiles, and interquartile range for each unique inter-anatomical spatial relationship. In one embodiment, the system computes the first quartile (Q1) as the 25th percentile, the median as the 50th percentile, and the third quartile (Q3) as the 75th percentile of the distribution of values for each spatial relationship. The interquartile range (IQR) is calculated as the difference between Q3 and Q1, providing a measure of variability in the relationship. In another embodiment, the system may determine thresholds by setting the upper threshold to a sum of the median value and a predetermined multiple of the interquartile range, and setting the lower threshold to a difference between the median value and the predetermined multiple of the interquartile range. This approach provides an alternative statistical method for establishing monitoring thresholds that accounts for both the central tendency and spread of the spatial relationship measurements. In yet another embodiment, the system may calculate additional statistical measures such as mean, standard deviation, or percentile ranges to characterize the distributions more comprehensively.

At operation 1114, the image processing system determines inner thresholds and outer thresholds using the calculated statistical measures. In one embodiment, the system sets inner thresholds at Q1−1.5(Q3−Q1) and Q3+1.5(Q3−Q1), representing boundaries for typical anatomical relationships, while outer thresholds are set at Q1−3(Q3−Q1) and Q3+3(Q3−Q1), representing boundaries for anatomically plausible relationships. In another embodiment, the system may determine thresholds using a machine learning model trained on a large dataset of anatomical relationships to learn appropriate threshold values that maximize detection of anomalous relationships while minimizing false positives. In some embodiments, the system may employ an adaptive thresholding approach where the multipliers applied to the IQR are automatically adjusted based on the observed variance in the specific type of anatomical relationship being analyzed-using smaller multipliers for relationships that typically show less natural variation and larger multipliers for relationships with greater inherent variability. In some embodiments, the thresholds may be determined using a percentile-based approach, where inner thresholds are set at the 10th and 90th percentiles of the reference distribution, and outer thresholds are set at the 1st and 99th percentiles. In yet another embodiment, the system may employ a hybrid approach that combines multiple threshold determination methods, selecting the most appropriate thresholds based on factors such as the anatomical structures involved, the type of spatial measurement (angle vs. distance), and the clinical significance of potential deviations.

At operation 1116, the image processing system stores the statistical measures and inner thresholds and outer thresholds for each unique inter-anatomical spatial relationship in a database. In one embodiment, the system saves the measures and thresholds in a structured format that associates them with specific pairs of anatomical elements while excluding protected health information, enabling their use for monitoring spatial relationships between anatomical landmarks. In another embodiment, the system may store multiple sets of thresholds derived using different statistical approaches, allowing selection of the most appropriate thresholds for different types of spatial measurements or clinical applications.

Following operation 1116, method 1100 may end. Method 1100 provides significant computational advantages by transforming complex anatomical relationships into compact statistical representations that enable efficient monitoring of deployed machine learning models. By computing statistical measures and thresholds from normalized measurements across a reference dataset, the method eliminates the need to store or transmit actual medical images while still capturing the salient characteristics of anatomical relationships. The hierarchical threshold approach, using both inner and outer bounds, provides greater sensitivity in detecting subtle deviations from expected relationships while maintaining specificity through the outer threshold criteria. Additionally, the method's use of normalized measurements makes the statistical measures robust across different patient populations without requiring complex registration or standardization of the underlying medical images.

Referring to FIG. 12, a flowchart of a method 1200 for processing inter-anatomical spatial relationship data from medical images is shown. Method 1200 enables efficient statistical analysis of spatial relationships between anatomical landmarks by identifying and removing outliers from reference datasets, resulting in more reliable thresholds for monitoring deployed machine learning model performance.

At operation 1202, the image processing system retrieves normalized distance and angle values for all medical images in the reference dataset. In one embodiment, for each unique inter-anatomical spatial relationship, the system accesses previously determined normalized distances between centers of anatomical landmarks and angles between orientation vectors of anatomical planes. For example, when analyzing knee imaging data, the system may retrieve normalized distances between the centers of the lateral and medial menisci, and angles between the tibial plateau plane and femoral shaft axis. In another embodiment, the system retrieves spatial relationships between organ masks and bounding boxes by accessing normalized measurements of distances between their centers, edges, or other geometric features, such as distances between vertebral body centers in spine imaging.

At operation 1204, the image processing system calculates initial statistical measures including median, first quartile (Q1), and third quartile (Q3) for the retrieved values. In one embodiment, the system computes these measures for each unique spatial relationship type across all medical images in the reference dataset. For example, when analyzing relationships between optic nerve landmarks, the system calculates the median, Q1, and Q3 values for both the normalized distances between nerve endpoints and the angles between fitted nerve planes. In another embodiment, the statistical measures are calculated separately for different anatomical regions or imaging protocols to account for variations in expected spatial relationships.

At operation 1206, the image processing system determines the interquartile range (IQR) by subtracting Q1 from Q3. In one embodiment, the IQR calculation provides a measure of statistical dispersion for each spatial relationship type, enabling identification of outliers based on the spread of typical values in the reference dataset. For example, when analyzing distances between vertebral landmarks, the IQR represents the range within which the middle 50% of all normalized distance measurements fall. In another embodiment, the system may calculate multiple IQR values for different subsets of the data, such as separate IQRs for different patient demographics or anatomical variations.

At operation 1208, the image processing system calculates outer fences as (Q1−3*IQR) and (Q3+3*IQR). In one embodiment, these outer fences establish boundaries for identifying extreme outliers in the spatial relationship measurements, with the factor of 3 providing a conservative threshold for outlier detection. For example, when analyzing angles between ankle joint planes, measurements falling beyond three times the IQR below Q1 or above Q3 would be considered extreme outliers. In another embodiment, the system may use different multipliers for calculating the outer fences based on the specific requirements of different anatomical regions or clinical applications.

At operation 1210, the image processing system identifies outliers as values falling outside the outer fences. In one embodiment, the system flags any normalized distance or angle measurements that exceed either the upper or lower outer fence for their respective spatial relationship type. For example, when analyzing relationships between shoulder joint landmarks, measurements such as the glenohumeral joint spacing or scapular plane angles that fall beyond the outer fences would be identified as outliers. In another embodiment, the system may employ additional criteria for outlier identification, such as considering the frequency of outlier occurrences for specific anatomical landmarks.

At operation 1212, the image processing system removes outliers from the dataset. In one embodiment, the system creates a cleaned dataset by excluding all measurements previously identified as outliers, while maintaining the association between remaining measurements and their corresponding anatomical landmarks. For example, when processing knee imaging data, outlier measurements of tibial-femoral alignment angles would be removed while preserving valid measurements for subsequent statistical analysis. In another embodiment, the system may maintain a separate record of removed outliers for quality control purposes or future analysis of systematic deviations. In one embodiment, the image processing system implements a hierarchical outlier removal process that considers both individual measurements and groups of related measurements. For example, when analyzing shoulder joint relationships, if multiple spatial measurements involving the glenohumeral joint are identified as outliers, the system may remove all measurements involving that anatomical landmark from the reference dataset, even if some individual measurements fall within the outer fences. In another embodiment, the system may apply different outlier removal criteria based on the anatomical context, such as using more conservative thresholds for pre-determined anatomical relationships or adjusting thresholds based on known anatomical variations. In one embodiment, the image processing system maintains a record of outlier removals to enable validation of the cleaning process and assessment of potential biases in the reference dataset. For example, when processing hip joint imaging data, the system may track the distribution of removed outlier measurements across different anatomical relationships to ensure the cleaning process does not systematically exclude certain types of valid anatomical variations. In another embodiment, the system may analyze patterns in removed outliers to identify potential correlations with specific imaging protocols or anatomical regions that might influence the reliability of the reference dataset used for establishing monitoring thresholds. The cleaned reference dataset enables more reliable statistical measures and thresholds for monitoring deployed model performance, as the removal of outliers reduces the impact of anomalous measurements on the derived statistical parameters. This improves the system's ability to detect genuine deviations in anatomical relationships during model deployment while maintaining appropriate sensitivity to normal anatomical variations.

At operation 1214, the image processing system recalculates statistical measures using the cleaned dataset with outliers removed. In one embodiment, the system computes updated values for the median, Q1, Q3, and IQR using only the remaining non-outlier measurements, providing more robust statistical measures for establishing monitoring thresholds. For example, when analyzing relationships between pelvic landmarks, the recalculated statistics would better reflect typical anatomical relationships by excluding extreme variations that might indicate incorrect landmark detection. In another embodiment, the system may calculate additional statistical measures such as skewness or kurtosis to characterize the distribution of the cleaned dataset.

At operation 1216, the image processing system stores the recalculated statistical measures and the inner and outer fences for the unique inter-anatomical spatial relationship. In one embodiment, the system saves these values in a structured format that associates each spatial relationship type with its corresponding statistical measures and threshold values, enabling efficient lookup during subsequent model monitoring. For example, when storing statistics for shoulder joint relationships, the system maintains separate entries for each unique combination of landmarks or planes, including their respective thresholds and distribution parameters. In another embodiment, the system may store multiple sets of statistical measures and thresholds corresponding to different patient populations or clinical contexts.

Following operation 1216, method 1200 may end. Method 1200 provides advantages by implementing an approach for establishing monitoring thresholds that are resistant to the influence of outliers in reference datasets. By systematically identifying and removing outlier measurements before calculating final statistical measures, the method produces more reliable thresholds for detecting anomalous spatial relationships in deployed model predictions. The iterative refinement of statistical measures through outlier removal helps ensure that the monitoring system can effectively distinguish between normal anatomical variations and potential model detection errors.

Referring to FIG. 13, a sagittal view medical diagnostic image 1300 of a human brain showing detected landmark planes is shown. The medical image 1300 comprises a grayscale magnetic resonance imaging (MRI) scan displaying internal brain structures with overlaid geometric reference planes detected by a trained machine learning model. In one embodiment, the medical image 1300 represents a T2-weighted MRI sequence providing high contrast visualization of brain tissue, ventricles, and other neuroanatomical structures. In another embodiment, the medical image 1300 may be generated from other MRI sequences or imaging modalities capable of capturing detailed anatomical relationships between brain structures in a sagittal orientation.

A first detected landmark plane 1302 is shown overlaid on the medical image 1300, represented by a stepped or segmented line indicating an anomalous geometric relationship relative to expected anatomical orientations. In one embodiment, the first detected landmark plane 1302 corresponds to an anterior commissure-posterior commissure (ACPC) reference plane whose angular orientation exceeds predetermined statistical thresholds derived from a reference dataset of anatomical relationships. In another embodiment, the first detected landmark plane 1302 may represent other anatomical reference planes such as a mid-sagittal plane (MSP) or temporal lobe plane whose spatial relationship with other detected landmarks has been flagged as an outlier based on normalized distance and angle measurements.

A second detected landmark plane 1304 is also shown overlaid on the medical image 1300, represented by a dashed line indicating a non-anomalous geometric relationship within expected anatomical parameters. In one embodiment, the second detected landmark plane 1304 corresponds to an optic nerve reference plane whose angular orientation and normalized distance relationships with other detected anatomical landmarks fall within predetermined statistical thresholds. In another embodiment, the second detected landmark plane 1304 may represent other anatomical reference planes such as a hippocampal plane or pituitary plane whose spatial relationships satisfy expected geometric constraints based on the statistical distribution of previously determined anatomical relationships.

The relative positioning and intersection of the first detected landmark plane 1302 and second detected landmark plane 1304 enables quantitative analysis of their spatial relationships, including normalized distances between their reference points and angles between their orientation vectors. In one embodiment, these geometric measurements are compared against predetermined upper and lower thresholds to identify potential anomalies in anatomical landmark detection. In another embodiment, the intersection patterns of the detected landmark planes may be analyzed to characterize biases or deviations in the machine learning model's performance across different deployment sites.

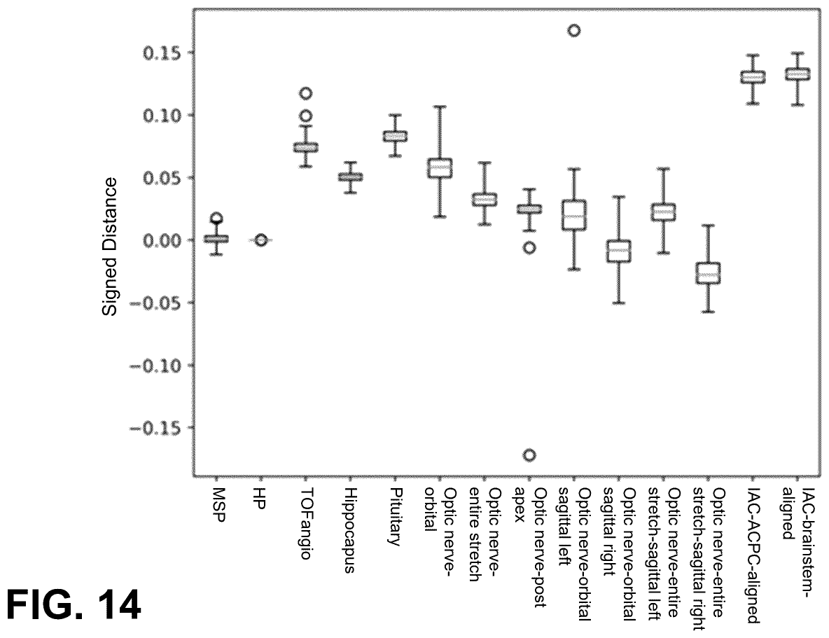

Referring to FIG. 14, a box plot depicting the distribution of signed distance measurements across various anatomical features is shown. The box plot enables visualization of statistical distributions of normalized spatial relationships between detected anatomical landmarks, planes, and organs identified in medical images by deployed machine learning models. The y-axis represents signed distance measurements ranging from approximately −0.15 to 0.15, while the x-axis displays multiple anatomical feature categories including mid-sagittal plane (MSP), hippocampus (HP), TOF angio, pituitary, optic nerve-related, and internal auditory canal (IAC) measurements.

For each anatomical feature category, the box plot shows the statistical distribution through several visual elements. The central box represents the interquartile range (IQR) containing the middle 50% of measurements, with the horizontal line within each box indicating the median value. In one embodiment, these distributions are derived from a reference dataset of previously determined spatial relationships, with the upper and lower bounds of the boxes corresponding to the third quartile (Q3) and first quartile (Q1) respectively. In another embodiment, the box boundaries represent inner thresholds used for classifying spatial relationships, where Q3+1.5(Q3−Q1) defines the upper threshold and Q−1.5(Q3−Q1) defines the lower threshold.

The whiskers extending from each box show the range of typical measurements falling within outer thresholds, defined in one embodiment as Q3+3(Q3−Q1) for the upper threshold and Q1−3(Q3−Q1) for the lower threshold. Individual points beyond the whiskers represent statistical outliers, indicating measurements that deviate significantly from expected anatomical relationships. The IAC-related measurements show notably higher positive signed distances around 0.13-0.15, while most central categories exhibit measurements clustered near zero, suggesting consistent detection of expected anatomical relationships in these regions.

The varying spread of values across different anatomical features, indicated by different box sizes and whisker lengths, reveals the relative variability in spatial relationships for different anatomical structures. Larger spreads may indicate anatomical relationships with greater natural variation across patient populations. Wider distributions may highlight areas where the machine learning model exhibits lower consistency in landmark detection. Several optic nerve-related categories show notable outliers, particularly in measurements involving orbital and sagittal plane relationships.

The signed nature of the distance measurements provides directionality information about spatial relationships. In one embodiment, positive values may indicate an anatomical landmark is positioned anterior or superior to its paired landmark, while negative values indicate posterior or inferior positioning. In another embodiment, the sign may represent deviations from expected anatomical relationships, with negative values indicating relationships that fall short of expected distances and positive values indicating relationships that exceed expected distances.

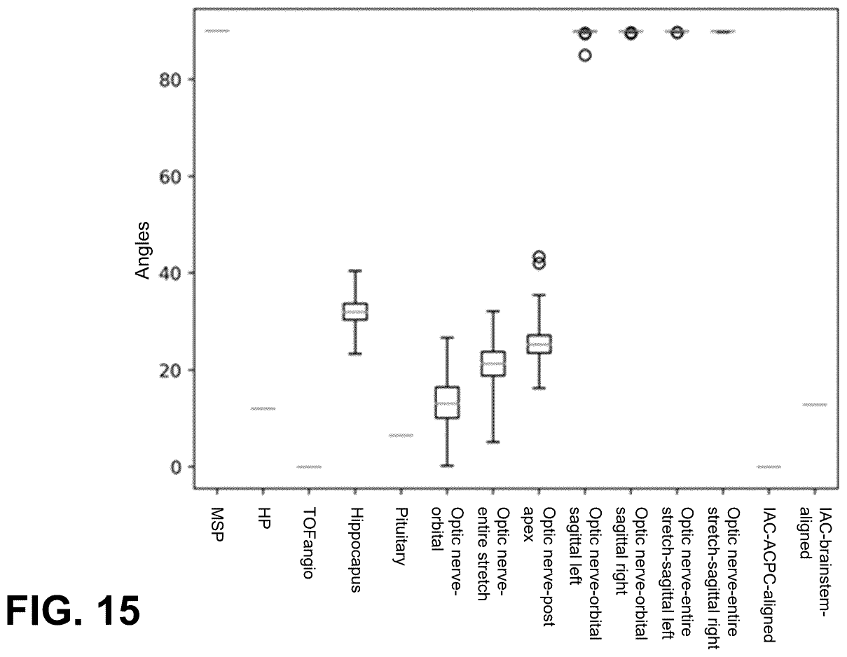

Referring to FIG. 15, a box plot showing the distribution of angular measurements across various anatomical imaging alignments and structures is shown. The box plot enables visualization of statistical distributions of angular relationships between detected anatomical landmarks, planes, and organs identified in medical images from a reference dataset. The y-axis represents angle measurements ranging from 0 to 80 degrees in 20-degree increments, while the x-axis displays multiple anatomical feature categories including mid-sagittal plane (MSP), hippocampus (HP), TOF angio, pituitary, optic nerve-related measurements, and internal auditory canal (IAC) alignments.

The angular measurements shown in the plot represent geometric relationships between different anatomical planes and structures. The angles correspond to the relative orientation between detected anatomical planes, such as the angle between the MSP and ACPC plane, which provides information about the symmetry and alignment of brain structures. In some embodiments, the angles represent the orientation of elongated structures like optic nerves relative to standard anatomical reference planes, enabling assessment of anatomical positioning across different patient populations.

The plot shows distinct patterns across different anatomical categories. The IAC measurements, including IAC-brainstem and IAC-ACPC alignments, demonstrate relatively consistent angular relationships with small variations, appearing as compact boxes with short whiskers. In one embodiment, these measurements reflect the expected geometric constraints between the internal auditory canal and neurological reference planes. In another embodiment, they provide statistical thresholds for detecting anomalous orientations that may indicate incorrect landmark detection by deployed machine learning models.

The optic nerve measurements show more complex distributions, particularly for the orbital and entire stretch categories. In one embodiment, the bilateral measurements (left and right) of orbital sagittal and entire stretch sagittal orientations enable assessment of symmetry in optic nerve positioning. In another embodiment, the post apex measurements provide reference angles for evaluating the geometric relationship between the posterior portion of the optic nerve and surrounding anatomical structures.

Additional anatomical structures, including the pituitary and hippocampus, show varying degrees of angular dispersion. In one embodiment, these measurements characterize the expected orientation relationships between these structures and standard anatomical planes like the MSP, establishing normal ranges for automated anomaly detection.

The box plot components, including median lines, interquartile ranges, whiskers, and outlier points, provide statistical characterization of the angular relationships. In one embodiment, these statistical measures serve as thresholds for detecting anomalous geometric relationships in newly acquired medical images. In another embodiment, they enable quantitative assessment of machine learning model performance by comparing detected anatomical relationships against the reference distribution patterns established from the dataset.

The disclosure also provides support for a method comprising: receiving a medical image at a clinical site, detecting a plurality of anatomical landmarks in the medical image using a deployed machine learning model at the clinical site, determining spatial relationships between pairs of the detected anatomical landmarks, transmitting the determined spatial relationships to a remote monitoring system without transmitting the medical image, comparing the determined spatial relationships against a plurality of previously determined spatial relationships to identify outlier relationships, generating a performance characterization of the deployed machine learning model based on the identified outlier relationships, and responding to the performance characterization indicating a deviation from expected model performance by transmitting an alert to a user device. In a first example of the method, determining spatial relationships between pairs of the detected anatomical landmarks comprises: determining angles between orientation vectors of anatomical landmark planes for each pair of detected anatomical landmarks, determining distances between centers of organ masks and segmentation masks for each pair of detected anatomical landmarks, and normalizing the distances with respect to an anatomical extent determined from the detected anatomical landmarks to account for variations in patient size. In a second example of the method, optionally including the first example, comparing the determined spatial relationships against the plurality of previously determined spatial relationships comprises: accessing a pre-determined upper threshold and a pre-determined lower threshold for each spatial relationship, wherein the upper threshold and lower threshold are derived from statistical distributions of previously determined spatial relationships in a reference dataset, and identifying as outliers any spatial relationships having values outside their respective upper and lower thresholds. In a third example of the method, optionally including one or both of the first and second examples, the upper and lower thresholds for each spatial relationship are determined by: computing statistical quartiles Q1 and Q3 from a distribution of values for that spatial relationship in the reference dataset, wherein Q1 is a 25th percentile and Q3 is a 75th percentile of the distribution of values, and setting the upper threshold to Q3+3(Q3−Q1) and the lower threshold to Q1−3(Q3−Q1). In a fourth example of the method, optionally including one or more or each of the first through third examples, detecting the plurality of anatomical landmarks in the medical image using the deployed machine learning model comprises one or more of: detecting organ masks delineating boundaries of anatomical organs, detecting segmentation masks identifying anatomical structures, detecting landmark masks indicating positions of anatomical reference points, detecting planes derived from the anatomical reference points, and detecting bounding boxes encompassing regions of interest. In a fifth example of the method, optionally including one or more or each of the first through fourth examples, the performance characterization includes: a list of anatomical landmark pairs having outlier spatial relationships, for each outlier spatial relationship, a determined angle and distance value and corresponding upper and lower thresholds, and a frequency of outlier relationships for each anatomical landmark. In a sixth example of the method, optionally including one or more or each of the first through fifth examples, comparing the determined spatial relationships comprises: identifying which spatial relationships between pairs of anatomical landmarks were successfully determined, comparing the successfully determined spatial relationships against their respective previously determined spatial relationships, and excluding from comparison any spatial relationships that could not be determined due to one or more anatomical landmarks not being detected in the medical image. In a seventh example of the method, optionally including one or more or each of the first through sixth examples, the alert includes: an identification of which anatomical landmarks are most frequently involved in outlier relationships, specific angle and distance measurements that exceeded thresholds, and a warning to carefully review any anatomical landmarks identified as anomalous before accepting results from the deployed machine learning model.

The disclosure also provides support for a system comprising: a first device located at a clinical site, wherein the first device comprises: a first non-transitory memory including a deployed machine learning model and instructions, and a first processor, wherein, when executing the instructions, the first processor causes the first device to: receive a medical image, detect a plurality of anatomical landmarks in the medical image using the deployed machine learning model, determine spatial relationships between pairs of the detected anatomical landmarks, and transmit the determined spatial relationships to a second device without transmitting the medical image, the second device located remotely from the first device, wherein the first device and the second device are communicatively coupled, and wherein the second device comprises: a second non-transitory memory including instructions, and a second processor, wherein, when executing the instructions, the second processor causes the second device to: receive the determined spatial relationships from the first device, compare the determined spatial relationships against a plurality of previously determined spatial relationships to identify outlier relationships, generate a performance characterization of the deployed machine learning model based on the identified outlier relationships, and respond to the performance characterization indicating a deviation from expected model performance by transmitting an alert to a user device. In a first example of the system, determining spatial relationships between pairs of the detected anatomical landmarks comprises: determining angles between orientation vectors of the pairs of anatomical landmarks, and determining normalized distances between centers of the pairs of anatomical landmarks, wherein the distances are normalized with respect to an anatomical size of an imaging subject. In a second example of the system, optionally including the first example, the first device comprises an image acquisition device configured to acquire the medical image, and wherein the first processor is configured to detect the plurality of anatomical landmarks in the medical image in real-time during image acquisition. In a third example of the system, optionally including one or both of the first and second examples, the second device comprises a remote server, and wherein the second device further comprises: a database storing the plurality of previously determined spatial relationships, and a dashboard interface configured to display statistics of the performance characterization across multiple clinical sites.

The disclosure also provides support for a method for monitoring performance of a deployed machine learning model, comprising: receiving, at a remote monitoring site, a plurality of spatial interrelationship measurements between anatomical landmarks detected by the deployed machine learning model in a medical image at a deployment site, wherein the spatial interrelationship measurements exclude the medical image and patient identifying information, and wherein the anatomical landmarks comprise one or more geometric objects including organ masks delineating boundaries of anatomical organs, segmentation masks identifying anatomical structures, landmark masks indicating positions of anatomical reference points, planes derived from the anatomical reference points, and bounding boxes encompassing regions of interest, accessing a reference dataset comprising spatial interrelationship measurements between corresponding anatomical landmarks detected in a plurality of training images, for each type of spatial interrelationship measurement between pairs of anatomical landmarks: determining statistical measures from spatial interrelationship measurements from the reference dataset of the type, determining an upper threshold and a lower threshold based on the statistical measures determined from the reference dataset, comparing each received spatial interrelationship measurement against the upper threshold and lower threshold determined for the type of spatial interrelationship measurement, determining a frequency of outlier occurrences for each anatomical landmark by counting a number of times the anatomical landmark appears in spatial interrelationship measurements outside the upper threshold and lower bound threshold, identifying one or more anomalous anatomical landmarks based on the frequency of outlier occurrences exceeding a predetermined frequency threshold, and transmitting an alert identifying the one or more anomalous anatomical landmarks to the deployment site. In a first example of the method, determining statistical measures from the spatial interrelationship measurements of the reference dataset comprises: determining a median value and an interquartile range for each type of spatial interrelationship measurement, and wherein determining the upper threshold and lower threshold comprises: setting the upper threshold to a sum of the median value and a predetermined multiple of the interquartile range, and setting the lower threshold to a difference between the median value and the predetermined multiple of the interquartile range. In a second example of the method, optionally including the first example, the spatial interrelationship measurements comprise: angles between planes defined by detected anatomical landmarks, and normalized distances between centers of detected anatomical landmarks, wherein the distances are normalized based on an anatomical size measurement. In a third example of the method, optionally including one or both of the first and second examples, normalizing the distances between centers of detected anatomical landmarks comprises: determining a bounding box containing an anatomical region of interest in the medical image, determining dimensions of the bounding box, and dividing each distance measurement by a largest dimension of the bounding box to produce normalized distance measurements that are independent of patient size. In a fourth example of the method, optionally including one or more or each of the first through third examples, normalizing the distances between centers of detected anatomical landmarks comprises: determining an extent of an anatomical structure captured in the medical image, computing a reference anatomical size measurement based on the determined extent, and dividing each distance measurement by the reference anatomical size measurement to normalize each distance measurement relative to an corresponding anatomical structure size. In a fifth example of the method, optionally including one or more or each of the first through fourth examples, the method further comprises: receiving an indication of which anatomical landmarks were selected by a user at the deployment site for prescribing a scan plane, and in response to determining that an anatomical landmark selected by the user matches one of the identified anomalous anatomical landmarks, including a warning in the alert prompting the user to review the scan plane. In a sixth example of the method, optionally including one or more or each of the first through fifth examples, the alert includes: identities of the one or more anomalous anatomical landmarks, the frequency of outlier occurrences for each of the one or more anomalous anatomical landmarks, and spatial interrelationship measurements that exceeded the upper threshold or subceeded the lower threshold for each of the one or more anomalous anatomical landmarks. In a seventh example of the method, optionally including one or more or each of the first through sixth examples, the method further comprises: accumulating the received spatial interrelationship measurements over time for the deployment site, analyzing the accumulated measurements to identify systematic biases in detection of specific anatomical landmarks at the deployment site, and providing feedback regarding the identified systematic biases to the deployment site.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method comprising:
receiving a medical image at a clinical site;
detecting a plurality of anatomical landmarks in the medical image using a deployed machine learning model at the clinical site;
determining spatial relationships between pairs of the detected anatomical landmarks;
transmitting the determined spatial relationships to a remote monitoring system without transmitting the medical image;
comparing the determined spatial relationships against a plurality of previously determined spatial relationships to identify outlier relationships;
generating a performance characterization of the deployed machine learning model based on the identified outlier relationships; and
responding to the performance characterization indicating a deviation from expected model performance by transmitting an alert to a user device.

2. The method of claim 1, wherein determining spatial relationships between pairs of the detected anatomical landmarks comprises:
determining angles between orientation vectors of anatomical landmark planes for each pair of detected anatomical landmarks;
determining distances between centers of organ masks and segmentation masks for each pair of detected anatomical landmarks; and
normalizing the distances with respect to an anatomical extent determined from the detected anatomical landmarks to account for variations in patient size.

3. The method of claim 1, wherein comparing the determined spatial relationships against the plurality of previously determined spatial relationships comprises:
accessing a pre-determined upper threshold and a pre-determined lower threshold for each spatial relationship, wherein the upper threshold and lower threshold are derived from statistical distributions of previously determined spatial relationships in a reference dataset; and
identifying as outliers any spatial relationships having values outside their respective upper and lower thresholds.

4. The method of claim 3, wherein the upper and lower thresholds for each spatial relationship are determined by:
computing statistical quartiles Q1 and Q3 from a distribution of values for that spatial relationship in the reference dataset, wherein Q1 is a 25th percentile and Q3 is a 75th percentile of the distribution of values; and
setting the upper threshold to Q3+3(Q3−Q1) and the lower threshold to Q1−3(Q3−Q1).

5. The method of claim 1, wherein detecting the plurality of anatomical landmarks in the medical image using the deployed machine learning model comprises one or more of:
detecting organ masks delineating boundaries of anatomical organs;
detecting segmentation masks identifying anatomical structures;
detecting landmark masks indicating positions of anatomical reference points;
detecting planes derived from the anatomical reference points; and
detecting bounding boxes encompassing regions of interest.

6. The method of claim 1, wherein the performance characterization includes:
a list of anatomical landmark pairs having outlier spatial relationships;
for each outlier spatial relationship, a determined angle and distance value and corresponding upper and lower thresholds; and
a frequency of outlier relationships for each anatomical landmark.

7. The method of claim 1, wherein comparing the determined spatial relationships comprises:
identifying which spatial relationships between pairs of anatomical landmarks were successfully determined;
comparing the successfully determined spatial relationships against their respective previously determined spatial relationships; and
excluding from comparison any spatial relationships that could not be determined due to one or more anatomical landmarks not being detected in the medical image.

8. The method of claim 1, wherein the alert includes:
an identification of which anatomical landmarks are most frequently involved in outlier relationships;
specific angle and distance measurements that exceeded thresholds; and
a warning to carefully review any anatomical landmarks identified as anomalous before accepting results from the deployed machine learning model.

9. A system comprising:
a first device located at a clinical site, wherein the first device comprises:
a first non-transitory memory including a deployed machine learning model and instructions; and
a first processor, wherein, when executing the instructions, the first processor causes the first device to:
receive a medical image;
detect a plurality of anatomical landmarks in the medical image using the deployed machine learning model;
determine spatial relationships between pairs of the detected anatomical landmarks; and
transmit the determined spatial relationships to a second device without transmitting the medical image;
the second device located remotely from the first device, wherein the first device and the second device are communicatively coupled, and wherein the second device comprises:
a second non-transitory memory including instructions; and
a second processor, wherein, when executing the instructions, the second processor causes the second device to:
receive the determined spatial relationships from the first device;
compare the determined spatial relationships against a plurality of previously determined spatial relationships to identify outlier relationships;
generate a performance characterization of the deployed machine learning model based on the identified outlier relationships; and
respond to the performance characterization indicating a deviation from expected model performance by transmitting an alert to a user device.

10. The system of claim 9, wherein determining spatial relationships between pairs of the detected anatomical landmarks comprises:
determining angles between orientation vectors of the pairs of anatomical landmarks; and
determining normalized distances between centers of the pairs of anatomical landmarks, wherein the distances are normalized with respect to an anatomical size of an imaging subject.

11. The system of claim 9, wherein the first device comprises an image acquisition device configured to acquire the medical image, and wherein the first processor is configured to detect the plurality of anatomical landmarks in the medical image in real-time during image acquisition.

12. The system of claim 9, wherein the second device comprises a remote server, and wherein the second device further comprises:
a database storing the plurality of previously determined spatial relationships; and
a dashboard interface configured to display statistics of the performance characterization across multiple clinical sites.

13. A method for monitoring performance of a deployed machine learning model, comprising:
receiving, at a remote monitoring site, a plurality of spatial interrelationship measurements between anatomical landmarks detected by the deployed machine learning model in a medical image at a deployment site, wherein the spatial interrelationship measurements exclude the medical image and patient identifying information, and wherein the anatomical landmarks comprise one or more geometric objects including organ masks delineating boundaries of anatomical organs, segmentation masks identifying anatomical structures, landmark masks indicating positions of anatomical reference points, planes derived from the anatomical reference points, and bounding boxes encompassing regions of interest;
accessing a reference dataset comprising spatial interrelationship measurements between corresponding anatomical landmarks detected in a plurality of training images;
for each type of spatial interrelationship measurement between pairs of anatomical landmarks:
determining statistical measures from spatial interrelationship measurements from the reference dataset of the type;
determining an upper threshold and a lower threshold based on the statistical measures determined from the reference dataset;
comparing each received spatial interrelationship measurement against the upper threshold and lower threshold determined for the type of spatial interrelationship measurement;
determining a frequency of outlier occurrences for each anatomical landmark by counting a number of times the anatomical landmark appears in spatial interrelationship measurements outside the upper threshold and lower bound threshold;
identifying one or more anomalous anatomical landmarks based on the frequency of outlier occurrences exceeding a predetermined frequency threshold; and
transmitting an alert identifying the one or more anomalous anatomical landmarks to the deployment site.

14. The method of claim 13, wherein determining statistical measures from the spatial interrelationship measurements of the reference dataset comprises:
determining a median value and an interquartile range for each type of spatial interrelationship measurement; and
wherein determining the upper threshold and lower threshold comprises:
setting the upper threshold to a sum of the median value and a predetermined multiple of the interquartile range; and
setting the lower threshold to a difference between the median value and the predetermined multiple of the interquartile range.

15. The method of claim 13, wherein the spatial interrelationship measurements comprise:
angles between planes defined by detected anatomical landmarks; and
normalized distances between centers of detected anatomical landmarks, wherein the distances are normalized based on an anatomical size measurement.

16. The method of claim 15, wherein normalizing the distances between centers of detected anatomical landmarks comprises:
determining a bounding box containing an anatomical region of interest in the medical image;
determining dimensions of the bounding box; and
dividing each distance measurement by a largest dimension of the bounding box to produce normalized distance measurements that are independent of patient size.

17. The method of claim 15, wherein normalizing the distances between centers of detected anatomical landmarks comprises:
determining an extent of an anatomical structure captured in the medical image;
computing a reference anatomical size measurement based on the determined extent; and
dividing each distance measurement by the reference anatomical size measurement to normalize each distance measurement relative to an corresponding anatomical structure size.

18. The method of claim 13, further comprising:
receiving an indication of which anatomical landmarks were selected by a user at the deployment site for prescribing a scan plane; and
in response to determining that an anatomical landmark selected by the user matches one of the identified anomalous anatomical landmarks, including a warning in the alert prompting the user to review the scan plane.

19. The method of claim 13, wherein the alert includes:
identities of the one or more anomalous anatomical landmarks;
the frequency of outlier occurrences for each of the one or more anomalous anatomical landmarks; and
spatial interrelationship measurements that exceeded the upper threshold or subceeded the lower threshold for each of the one or more anomalous anatomical landmarks.

20. The method of claim 13, further comprising:
accumulating the received spatial interrelationship measurements over time for the deployment site;
analyzing the accumulated measurements to identify systematic biases in detection of specific anatomical landmarks at the deployment site; and
providing feedback regarding the identified systematic biases to the deployment site.
